(19) United States
(12) Patent Application Publication
(10) Pub. No.: US 2024/0400685 A1
(43) Pub. Date: Dec. 5, 2024

(54) HIGHLY PURIFIED EPCORITAMAB COMPOSITIONS

(71) Applicant: GENMAB A/S, Valby (DK)

(72) Inventors: Richard G. HIBBERT, GN Wassenaar (NL); Bolette BJERREGAARD, Espergaerde (DK); Søren S. JENSEN, Frederiksberg C (DK); César Filipe OLIVEIRA DA COSTA, Soesterberg (NL); Rohan O'DONNELL, Ballerup (DK)

(21) Appl. No.: 18/664,757

(22) Filed: May 15, 2024

(30) Foreign Application Priority Data
May 15, 2023 (EP) 23173500.2

(51) Int. Cl. C07K 16/28; C07K 1/18; C07K 1/34

(52) U.S. Cl. CPC C07K 16/2809; C07K 1/18; C07K 1/34; C07K 16/2887; C07K 2317/31

(57) ABSTRACT

The present disclosure provides highly purified epcoritamab compositions, which comprise a bispecific antibody that binds CD3 and CD20. In the highly purified compositions, impurities, such as epcoritamab protein variants, process-related impurities and/or product-related impurities, have been minimized or removed. Methods of preparing the highly purified epcoritamab compositions are also provided.

### FIELD

The present invention relates to compositions of epcoritamab, a bispecific antibody targeting both CD3 and CD20, that have been highly purified to minimize undesired protein variants, as well as process-related and/or product-related impurities. Methods of preparing the highly purified epcoritamab compositions are also provided.

### BACKGROUND

CD3 has been known for many years and therefore has been subject of interest in many aspects. Specifically, antibodies raised against CD3 or the T-cell Receptor Complex, which CD3 is part of, are known. An in vitro characterization of five humanized OKT3 effector function variant antibodies has been described (Xu et al., 2000, Cell Immunol. 200 (1): 16-26).

Treatment with the anti-CD3 monoclonal antibody hOKT3gamma1 (Ala-Ala) results in improved C-peptide responses and clinical parameters for at least 2 years after onset of type 1 diabetes in absence of continued immunosuppressive medications (Herold et al., 2005, Diabetes, 54 (6): 1763-9).

A promising approach to improve targeted antibody therapy is by delivering cytotoxic cells specifically to the antigen-expressing cancer cells. This concept of using T-cells for efficient killing of tumor cells has been described in Staerz, et. al., 1985, Nature 314:628-631). However, initial clinical studies were rather disappointing mainly due to low efficacy, severe adverse effects (cytokine storm) and immunogenicity of the bispecific antibodies (Muller and Kontermann, 2010, BioDrugs 24:89-98). Advances in the design and application of bispecific antibodies have partially overcome the initial barrier of cytokine storm and improved clinical effectiveness without dose-limiting toxicities (Garber, 2014, Nat. Rev. Drug Discov. 13:799-801; Lum and Thakur, 2011, BioDrugs 25:365-379). Critical to overcome the initial barrier of cytokine storm as described for catumaxomab (Berek et al. 2014, Int. J. Gynecol. Cancer 24 (9): 1583-1589; Mau-Sørensen et al. 2015, Cancer Chemother. Pharmacol. 75:1065-1073) was the absence or silencing of the Fc domain.

The CD20 molecule (also called human B-lymphocyte-restricted differentiation antigen or Bp35) is a hydrophobic transmembrane protein with a molecular weight of approximately 35 kD located on pre-B and mature B lymphocytes (Valentine et al. (1989) J. Biol. Chem. 264 (19): 11282-11287; and Einfield et al., (1988) EMBO J. 7 (3): 711-717). CD20 is found on the surface of greater than 90% of B cells from peripheral blood or lymphoid organs and is expressed during early pre-B cell development and remains until plasma cell differentiation. CD20 is present on both normal B cells as well as malignant B cells. In particular, CD20 is expressed on greater than 90% of B cell non-Hodgkin's lymphomas (NHL) (Anderson et al. (1984) Blood 63 (6): 1424-1433), but is not found on hematopoietic stem cells, pro-B cells, normal plasma cells, or other normal tissues (Tedder et al. (1985) J. Immunol. 135 (2): 973-979).

Methods for treating cancer as well as autoimmune and immune diseases by targeting CD20 are known in the art. For example, the chimeric CD20 antibody rituximab has been used for or suggested for use in treating cancers such as non-Hodgkin's lymphoma (NHL), chronic lymphocytic leukemia (CLL) and small lymphocytic lymphoma (SLL). The human monoclonal CD20 antibody ofatumumab has been used for or suggested for use in treating among others various CLL indications, follicular lymphoma (FL), neuromyelitis optica (NMO), diffuse and relapsing-remitting multiple sclerosis (RRMS). The human monoclonal CD20 antibody obinutuzumab has been used for or suggested for use in treating CLL. Furthermore, the humanized CD20 antibody ocrelizumab is being developed for RRMS.

Bispecific antibodies that bind to both CD3 and CD20 may be useful in therapeutic settings in which specific targeting and T cell-mediated killing of cells that express CD20 is desired. CD3×CD20 bispecific antibodies have been described in the art, for example in Hutchings et al. (2021) Lancet 398:1157-1169; Gall et al. (2005) Experimental Hematology 33:452; Stanglmaier et al. (2008) Int. J. Cancer: 123, 1181; Wu et al. (2007) Nat Biotechnol. 25:1290-1297; Sun et al. (2015) Science Translational Medicine 7, 287ra70; U.S. Pat. No. 10,544,220; US 2021/0371538; WO2011014659; WO2011090762; WO2011028952; WO2014047231; WO 2016/110576; and WO 2021/224499. While certain advances have been made, additional compositions, in particular those suitable for therapeutic use, are still needed.

### SUMMARY

The present disclosure pertains to highly purified compositions of the CD3×CD20 bispecific antibody epcoritamab. The compositions of the disclosure have minimized amounts of impurities, e.g., protein variants of epcoritamab and/or components remaining from the manufacturing process that potentially could interfere with the biological activity of epcoritamab compositions. As described herein, undesired protein variants of epcoritamab have been identified, including high molecular weight species (HMWS), low molecular weight species (LMWS), acidic species (e.g., deamidated species) and oxidized species. Moreover, product-related and process-related impurities have been identified. Epcoritamab compositions have now been obtained in which the presence of such impurities has been minimized, e.g., greatly reduced or eliminated. Furthermore, methods of preparing such compositions are also provided herein.

As further described in the Examples, potentially detrimental impurities in epcoritamab compositions have been identified, including high molecular weight species (HMWS) arising through aggregation, low molecular weight species (LMWS) arising through fragmentation, acidic species arising through deamidation at certain residues and oxidized species arising through oxidation at certain residues. Moreover, certain impurities were shown to have a detrimental effect on the biological activity of epcoritamab in T cell activation assays. Non-limiting examples include an HMWS dimer of epcoritamab comprising a signal sequence shown in SEQ ID NO: 9, a LMWS fragment of epcoritamab that is an Fc+Fab fragment, and an acidic variant of epcoritamab comprising deamidation at residues N103 and N106 in the heavy chain of the CD3 binding arm of epcoritamab. Thus, the compositions and methods of the disclosure provide means to minimize, e.g., reduce or eliminate, the presence of such impurities in epcoritamab compositions, including impurities that could potentially affect biological activity, which is of significant importance for therapeutic use.

Accordingly, in one aspect, the disclosure pertains to a composition comprising at least 95% epcoritamab and less than 5% of an impurity. In embodiments, epcoritamab comprises: (i) a CD3 binding arm comprising a heavy chain sequence as shown in SEQ ID NO: 1 and a light chain sequence as shown in SEQ ID NO: 2; and (ii) a CD20 binding arm comprising a heavy chain sequence as shown in SEQ ID NO: 3 and a light chain sequence as shown in SEQ ID NO: 4. In embodiments, the composition comprises less than 4%, 3%, 2%, 1% or lower of the impurity, or any one of the possible levels for the impurity as described in detail herein.

In embodiments, the impurity is a product-related impurity. In embodiments, the product-related impurity is selected from the group consisting of a high molecular weight species (HMWS) of epcoritamab, a low molecular weight species (LMWS) of epcoritamab, a charge variant of epcoritamab, an acidic species of epcoritamab, a basic species of epcoritamab, a deamidated species of epcoritamab, a glycosylated species of epcoritamab, a glycated species of epcoritamab, an oxidized species of epcoritamab, an N-terminal modification of epcoritamab, an aggregate of epcoritamab, a fragment of epcoritamab, a residual signal peptide modification of epcoritamab, an Fc fragment of epcoritamab and a Fab fragment of epcoritamab.

In embodiments, the product-related impurity is a high molecular weight species (HMWS) of epcoritamab. In embodiments, the HMWS is an epcoritamab dimer comprising a signal sequence as shown in SEQ ID NO: 9.

In embodiments, the product-related impurity is a low molecular weight species (LMWS) of epcoritamab. In embodiments, the LWMS is an Fc+Fab fragment of epcoritamab.

In embodiments, the product-related impurity is a deamidated acidic species of epcoritamab. In embodiments, the deamidated acidic species of epcoritamab comprises deamidation of one or more residues selected from the group consisting of a N103 residue in the heavy chain of the CD3 binding arm of epcoritamab, a N106 residue in the heavy chain of the CD3 binding arm of epcoritamab, a N103 and a N106 residues in the heavy chain of the CD3 binding arm of epcoritamab, a N333 residue in the heavy chain of the CD3 binding arm chain of epcoritamab, a N330 residue in the heavy chain of the CD20 binding arm of epcoritamab, and combinations thereof.

In embodiments, the impurity is a process-related impurity. In embodiments, the process-related impurity is selected from the group consisting of a reducing agent, an oxidizing agent, a manufacturing intermediate, a residual IgG1 homodimer intermediate, a residual host cell protein, a host cell nucleic acid, a media component and a chromatographic material.

In embodiments, the process-related impurity is a reducing agent. In embodiments, the reducing agent is 2-mercaptoethylamine (2-MEA). In embodiments, 2-MEA is present in the composition at a level at least 10-fold lower than the Permitted Daily Exposure (PDE) for 2-MEA.

In embodiments, the process-related impurity is an oxidizing agent. In embodiments, the oxidizing agent is dehydro-L-Ascorbic Acid (dhAA). In embodiments, dhAA is present in the composition at a level at least 100-fold lower than the Permitted Daily Exposure (PDE) for dhAA.

In embodiments, the process-related impurity is a manufacturing intermediate of epcoritamab. In embodiments, the manufacturing intermediate is the 3005a antibody or a portion thereof. In embodiments, the manufacturing intermediate is the 3001d antibody or a portion thereof.

In embodiments, the product-related impurity is a glycosylated species of epcoritamab. In embodiments, the glycosylated species comprises glycosylation of one or more residues selected from the group consisting of a N106 residue in the heavy chain of the CD3 binding arm of epcoritamab, a N305 residue in the heavy chain of the CD3 binding arm of epcoritamab, a N302 residue in the heavy chain of the CD20 binding arm of epcoritamab, and combinations thereof.

In embodiments, the product-related impurity is a glycated species of epcoritamab. In embodiments, the glycated species comprises glycation of one or more residues selected from the group consisting of a K331 residue in the heavy chain of the CD20 binding arm of epcoritamab, a K334 residue in the heavy chain of the CD3 binding arm of epcoritamab, and combinations thereof.

In embodiments, the product-related impurity is an oxidized species of epcoritamab. In embodiments, the oxidized species comprises oxidation of one or more residues selected from the group consisting of a M363 residue in the heavy chain of the CD20 binding arm of epcoritamab, a M366 residue on the CD3 heavy chain of epcoritamab, and combinations thereof.

In embodiments, the process-related impurity is a residual IgG1 homodimer intermediate.

In embodiments, the product-related impurity is an N-terminal modification of epcoritamab. In embodiments, the N-terminal modification is selected from the group consisting of a glutamate to pyroglutamate conversion, a residual signal peptide attached to epcoritamab, and combinations thereof. In embodiments, the glutamate to pyroglutamate conversion occurs in at least one of a CD3 heavy chain of epcoritamab, a CD20 heavy chain of epcoritamab and a CD20 light chain of epcoritamab. In embodiments, the glutamine to pyroglutamate conversion occurs in the CD3 light chain of epcoritamab. In embodiments, the residual signal peptide comprises an amino acid sequence selected from the group consisting of (a) Ser-Glu-Ala and (b) Leu-Cys-Thr-Gly-Ser-Glu-Ala (SEQ ID NO: 9).

In another aspect, the disclosure pertains to a composition comprising at least 95% epcoritamab and less than 5% of an acidic epcoritamab variant that is deamidated at residues N103 and N106 of CD3 heavy chain of epcoritamab. In other embodiments, the composition comprises less than 4%, 3%, 2%, 1% or lower of the acidic variant, or any one of the possible levels for the acidic variant as described in detail herein.

In another aspect, the disclosure pertains to a composition comprising at least 95% epcoritamab and less than 5% of a high molecular weight species (HMWS) of epcoritamab, wherein the HMWS is an epcoritamab dimer comprising a signal sequence having the amino acid sequence shown in SEQ ID NO: 9. In other embodiments, the composition comprises less than 4%, 3%, 2%, 1% or lower of the HMWS, or any one of the possible levels for the HMWS as described in detail herein.

In another aspect, the disclosure pertains to a composition comprising at least 95% epcoritamab and less than 5% of a low molecular weight species (LMWS) of epcoritamab, wherein the LMWS is an Fc+Fab fragment of epcoritamab. In other embodiments, the composition comprises less than 4%, 3%, 2%, 1% or lower of the LMWS, or any one of the possible levels for the LMWS as described in detail herein.

In embodiments, the composition is a pharmaceutical composition. In embodiments, the composition is a stable liquid composition suitable for subcutaneous administration. In embodiments, the composition comprises about 4 to 5 mg/mL of epcoritamab. In embodiments, the composition comprises about 60 mg/mL of epcoritamab.

In another aspect, the disclosure pertains to methods of preparing a composition of the disclosure. In an embodiment, the method comprises:
(a) combining a first binding arm of epcoritamab and a second binding arm of epcoritamab to form a combined sample;
(b) exposing the combined sample to reducing conditions to form a reduced combined sample;
(c) treating the reduced combined sample with an oxidizing agent to form an oxidized sample; and
(d) subjecting the oxidized sample to cation exchange chromatography such that the composition is prepared.

In embodiments, the combined sample is pH-adjusted to pH 7.0-7.7 before exposure to the reducing conditions. In embodiments, ultrafiltration/diafiltration (UF/DF) is performed after each of steps (b), (c) and (d). Additional details and components of the methods are described herein.

### BRIEF DESCRIPTION OF DRAWINGS

FIG. 1 is a schematic diagram of the CD3×CD20 bispecific antibody epcoritamab. Dotted lines=inter-disulfide bonds, asterisks=intra-disulfide bonds, black circles=N-linked glycosylation site, and F405L/K409R=Duobody® mutations

FIG. 2A and FIG. 2B are schematic diagrams of the CD3 binding arm 3005a (FIG. 2A) and CD20 binding arm 3001d (FIG. 2B) of epcoritamab. Dotted lines=inter-disulfide bonds, asterisks=intra-disulfide bonds, black circles=N-linked glycosylation site, and F405L/K409R=Duobody® mutations

FIG. 3A and FIG. 3B are schematic diagrams of purification process workflows for the CD3 binding arm (3005a) (FIG. 3A) and CD20 binding arm (3001d) (FIG. 3B) of epcoritamab.

FIG. 4 is a schematic diagram of a purification process workflow for bispecific antibody epcoritamab.

FIG. 5A and FIG. 5B are graphs showing representative dose response curves for epcoritamab in a CD4+CD25+ T cell activation assay (FIG. 5A) and a CD8+CD25+ T cell activation assay (FIG. 5B).

FIG. 6 shows a representative chromatogram of an SE-HPLC fractionation run.

FIG. 7 shows SE-HPLC chromatograms (zoomed view) of nominal, HMWS, main, and LMWS size fractions.

FIG. 8 shows an SEC-MALS chromatogram of HMWS fraction.

FIG. 9 shows an SEC-MALS chromatogram of LMWS fraction.

FIG. 10A and FIG. 10B show the distribution of species measured by SV-AUC at full scale (FIG. 10A) and zoomed in view (FIG. 10B).

FIG. 11A and FIG. 11B show electropherograms of non-reduced CE-SDS in nominal view (FIG. 11A) and zoomed in view (FIG. 11B).

FIG. 12A and FIG. 12B show electropherograms of reduced CE-SDS in nominal view (FIG. 12A) and zoomed in view (FIG. 12B).

FIG. 13 shows reduced CD-SDS electropherograms (zoomed view) of nominal, HMWS, main, and LMWS epcoritamab size fractions.

FIG. 14 shows non-reduced CD-SDS electropherograms (zoomed view) of nominal, HMWS, main, and LMWS epcoritamab size fractions.

FIG. 15 shows CIEX-HPLC chromatograms of charge fractions and nominal material.

### DETAILED DESCRIPTION

### Definitions

The term “immunoglobulin” as used herein refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized (see, e.g., Fundamental Immunology Ch. 7 (Paul, W., ed., 2nd ed. Raven Press, N.Y. (1989)). Briefly, each heavy chain typically is comprised of a heavy chain variable region (abbreviated herein as VH or V_{H}) and a heavy chain constant region (abbreviated herein as CH or C_{H}). The heavy chain constant region typically is comprised of three domains, CH1, CH2, and CH3. The hinge region is the region between the CH1 and CH2 domains of the heavy chain and is highly flexible. Disulfide bonds in the hinge region are part of the interactions between two heavy chains in an IgG molecule. Each light chain typically is comprised of a light chain variable region (abbreviated herein as VL or V_{L}) and a light chain constant region (abbreviated herein as CL or C_{L}). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J Mol Biol 1987; 196:90117). Unless otherwise stated or contradicted by context, CDR sequences herein are identified according to IMGT rules (Brochet X., Nucl Acids Res 2008; 36: W503-508; Lefranc M P., Nucl Acids Res 1999; 27:209-12; www.imgt.org/). Unless otherwise stated or contradicted by context, reference to amino acid positions in the constant regions is according to the EU-numbering (Edelman et al., PNAS. 1969; 63:78-85; Kabat et al., Sequences of Proteins of Immunological Interest, Fifth Edition. 1991 NIH Publication No. 91-3242). Unless otherwise stated or contradicted by context, reference to amino acid positions in the heavy and light variable region of the CD3 binding arm refers to their position in the amino acid sequences set forth in SEQ ID Nos: 1 and 2, respectively. Unless otherwise stated or contradicted by context, reference to amino acid positions in the heavy and light variable region of the CD20 binding arm refers to their position in the amino acid sequences set forth in SEQ ID Nos: 3 and 4, respectively.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is within the ability of one of ordinary skill in the art to align a sequence or segment in a sequence and thereby determine the corresponding position in a sequence to an amino acid position according to the present invention.

The term “antibody” (Ab) as used herein in the context of the present invention refers to an immunoglobulin molecule which has the ability to specifically bind to an antigen. The variable regions of the heavy and light chains of the immunoglobulin molecule contain a binding domain that interacts with an antigen. The term “antibody-binding region” or “antigen-binding region” as used herein refers to the region which interacts with the antigen and comprises both the VH and the VL regions.

The term “bispecific antibody” or “bs” or “bsAb” as used herein refers to an antibody having two different antigen-binding regions defined by different antibody sequences. A bispecific antibody can be of any format.

The terms “half molecule”, “Fab-arm”, and “arm”, as used herein, refer to one heavy chain-light chain pair.

The term “full-length” as used herein in the context of an antibody indicates that the antibody is not a fragment but contains all of the domains of the particular isotype normally found for that isotype in nature, e.g., the VH, CH1, CH2, CH3, hinge, VL and CL domains for an IgG1 antibody. A full-length antibody may be engineered. An example of a “full-length” antibody is epcoritamab.

The term “Fc region” as used herein refers to an antibody region consisting of the Fc sequences of the two heavy chains of an immunoglobulin, wherein said Fc sequences comprise at least a hinge region, a CH2 domain, and a CH3 domain.

The term “heterodimeric interaction between the first and second CH3 regions” as used herein refers to the interaction between the first CH3 region and the second CH3 region in a first-CH3/second-CH3 heterodimeric protein.

The term “homodimeric interactions of the first and second CH3 regions” as used herein refers to the interaction between a first CH3 region and another first CH3 region in a first-CH3/first-CH3 homodimeric protein and the interaction between a second CH3 region and another second CH3 region in a second-CH3/second-CH3 homodimeric protein.

The term “isolated antibody” as used herein refers to an antibody which is substantially free of other antibodies having different antigenic specificities. In a preferred embodiment, an isolated bispecific antibody that specifically binds to CD20 and CD3 is in addition substantially free of monospecific antibodies that specifically bind to CD20 or CD3.

The term “CD3” as used herein refers to the Cluster of Differentiation 3 protein, e.g., human CD3, which is part of the T-cell co-receptor protein complex and is composed of four distinct chains. In mammals, the complex contains a CD3γ (gamma) chain (human CD3γ chain UniProtKB/Swiss-Prot No P09693), a CD3δ (delta) chain (human CD3δ UniProtKB/Swiss-Prot No P04234), two CD3ε (epsilon) chains (human CD3ε UniProtKB/Swiss-Prot No P07766), and a CD3ζ-chain (zeta) chain (human CD3ζ UniProtKB/Swiss-Prot No P20963,). These chains associate with a molecule known as the T-cell receptor (TCR) and generate an activation signal in T lymphocytes. The TCR and CD3 molecules together comprise the TCR complex.

The term “CD3 antibody” or “anti-CD3 antibody” as used herein refers to an antibody which binds specifically to the antigen CD3, in particular human CD3δ (epsilon).

The term “human CD20” or “CD20” refers to human CD20 (UniProtKB/Swiss-Prot No P11836).

The term “CD20 antibody” or “anti-CD20 antibody” as used herein refers to an antibody which binds specifically to the antigen CD20, in particular to human CD20.

The term “CD3×CD20 antibody”, “anti-CD3×CD20 antibody”, “CD20×CD3 antibody” or “anti-CD20×CD3 antibody” as used herein refers to a bispecific antibody which comprises two different antigen-binding regions, one of which binds specifically to the antigen CD20 and one of which binds specifically to CD3.

The terms “epcoritamab” and “DuoBody-CD3×CD20” as used interchangeably herein refer to an IgG1 bispecific CD3×CD20 antibody comprising a first heavy and light chain pair as defined in SEQ ID NO: 1 and SEQ ID NO: 2, respectively, and comprising a second heavy and light chain pair as defined in SEQ ID NO: 3 and SEQ ID NO: 4. The first heavy and light chain pair comprises a region which binds to human CD3δ (epsilon), the second heavy and light chain pair comprises a region which binds to human CD20. The first binding region comprises the VH and VL sequences as defined by SEQ ID NOs: 5 and 6, and the second binding region comprises the VH and VL sequences as defined by SEQ ID NOs: 7 and 8. This bispecific antibody can be prepared as described in WO 2016/110576.

The term “reducing conditions” or “reducing environment” as used herein refers to a condition or an environment in which a substrate, here a cysteine residue in the hinge region of an antibody, is more likely to become reduced than oxidized.

The term “recombinant host cell” (or simply “host cell”) as used herein is intended to refer to a cell into which an expression vector has been introduced, e.g., an expression vector encoding an antibody described herein. Recombinant host cells include, for example, transfectomas, such as CHO, CHO-S, HEK, HEK293, HEK-293F, Expi293F, PER.C6 or NS0 cells, and lymphocytic cells.

The term “treatment” refers to the administration of an effective amount of a therapeutically active antibody described herein for the purpose of easing, ameliorating, arresting or eradicating (curing) symptoms or disease states such as DLBCL. Treatment may result in a complete response (CR), partial response (PR), or stable disease (SD), for example, as defined by Lugano criteria and/or LYRIC. Treatment may be continued, for example, for up to one year total duration of treatment with the bispecific antibody from initiation of R-CHOP, or up to disease progression or unacceptable toxicity.

The terms “administering”, “administered” or “administration” as used herein refer to the physical introduction of a composition (or formulation) comprising a therapeutic agent to a subject, using any suitable methods and delivery systems known to those skilled in the art. For example, an epcoritamab composition of the disclosure can be administered by a subcutaneous route.

The term “effective amount” or “therapeutically effective amount” refers to an amount effective, at dosages and for periods of time necessary, to achieve a desired therapeutic result. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects. In some embodiments, patients treated with the methods described herein will show an improvement in ECOG performance status.

The term “inhibits growth” of a tumor as used herein includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or 100%.

The term “subject” as used herein refers to a human patient, for example, a human patient with a tumor to be treated with a CD3×CD20 bispecific antibody. The terms “subject” and “patient” are used interchangeably herein.

As used herein, the term “about” refers to a value that is no more than 10% above and no more than 10% below a specified value.

As used herein, the term “impurity” refers to a substance potentially present within an epcoritamab composition but that differs from the epcoritamab bispecific antibody itself. An impurity can be an undesired or unwanted substance or component within the composition, for example due to its potential to interfere with epcoritamab activity. Non-limiting examples of impurities include protein variants of epcoritamab and substances that potentially remain in an epcoritamab sample after manufacturing. As used herein, a “product-related impurity” refers to an impurity that is an unwanted alteration or variant of the epcoritamab product in the sample, such as a chemical variant or breakdown product. As used herein, a “process-related impurity” refers to an impurity that results from a process (e.g., manufacturing process) that generates or involves the epcoritamab sample, such as unwanted residual reagents and contaminants. The amount of an impurity in a sample can be quantified by any one of various means established in the art, non-limiting examples of which include percentage purity/impurity in the sample, weight/volume of impurity in the sample and level below the Permitted Daily Exposure (PDE) of the impurity in the sample.

As used herein, “percentage impurity” or “percentage purity” refers to one possible means for quantifying the amount of an impurity or epcoritamab, respectively, in an epcoritamab composition. The % of an impurity in a sample can, for example, be calculated according to the formula: % impurity=mass of impurity in sample/total mass of sample×100. Similarly, the % of epcoritamab in a sample can, for example, be calculated according to the formula: % epcoritamab=mass of epcoritamab in a sample/total mass of sample×100. The % of an impurity in a sample can, for example, can also be calculated according to the formula: % impurity=volume of impurity in sample/total volume of sample×100. Similarly, the % of epcoritamab in a sample can, for example, be calculated according to the formula: % epcoritamab=volume of epcoritamab in a sample/total volume of sample×100.

All patent applications, patents, and printed publications referred to herein are incorporated by reference in their entirety to the same extent as if each individual publication, patent, or patent application was specifically and individually indicated to be incorporated by reference in its entirety. And, all patent applications, patents, and printed publications cited herein are incorporated herein by reference in their entireties, except for any definitions, subject matter disclaimers, or disavowals, and except to the extent that the incorporated material is inconsistent with the express disclosure herein, in which case the language in this disclosure controls.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate and are provided for description.

### Epcoritamab Structure and Variants

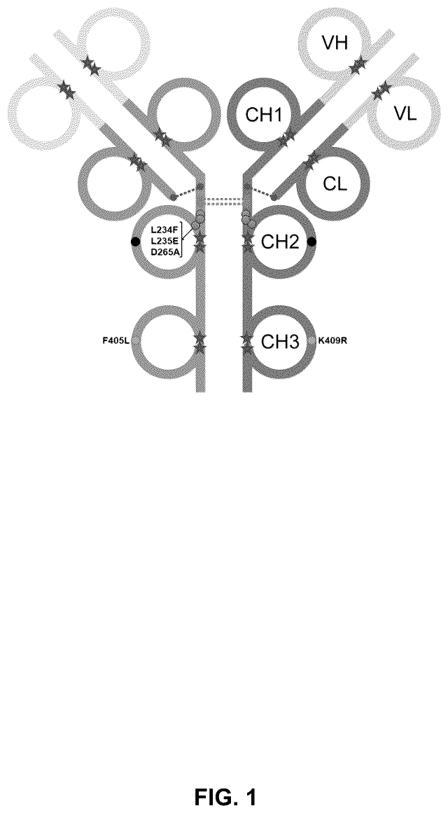

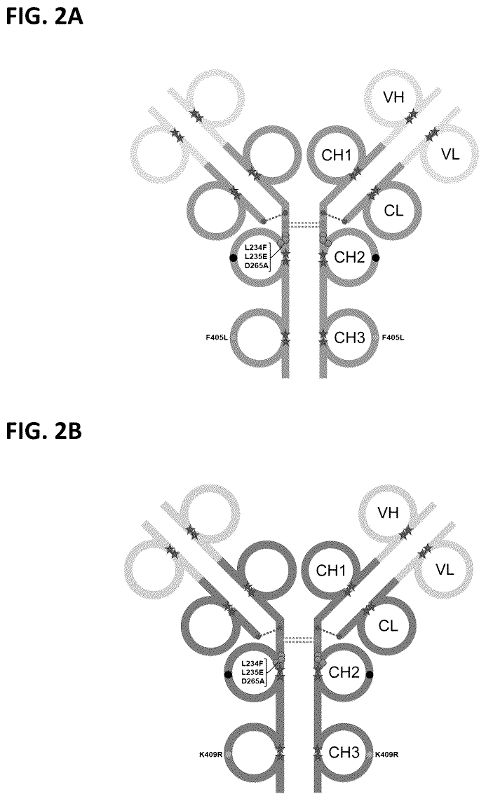

The structure of epcoritamab is illustrated schematically in FIG. 1 and described in detail in Example 1. The CD3 binding arm of epcoritamab is referred to as 3005a, which is illustrated schematically in FIG. 2A. The amino acid sequences of the 3005a full length heavy and light chains are shown in SEQ ID NOs: 1 and 2, respectively. The CD20 binding arm of epcoritamab is referred to as 3001d, which is illustrated schematically in FIG. 2B. The amino acid sequences of the 3001d full length heavy and light chains are shown in SEQ ID NOs: 3 and 4, respectively. The amino acid sequences of the VH and VL regions of 3005a are shown in SEQ ID NOs: 5 and 6, respectively. The amino acid sequences of the VH and VL regions of 3001d are shown in SEQ ID NOs: 7 and 8, respectively. Epcoritamab is also referred to herein as DuoBody CD3×CD20 and has CAS Registry number CAS 2134641-34-0.

As further described in the Examples, protein variants of epcoritamab have been identified, e.g., by forced degradation studies. These variants included high molecular weight species (HMWS) arising through aggregation, low molecular weight species (LMWS) arising through fragmentation, acidic species arising through deamidation at certain residues and oxidized species arising through oxidation at certain residues. Thus, these studies identified undesired components that potentially could be present in an epcoritamab composition and that potentially could interfere with the biological activity of the composition.

Accordingly, in embodiments, an impurity that is removed from the epcoritamab compositions of the disclosure can be, for example, an HMWS, an LMWS, an acidic species or an oxidized species.

In an embodiment, the acidic species is a deamidated variant of epcoritamab. In an embodiment, the deamidated variant of epcoritamab comprises deamidation of the N103 and N106 residues on the 3005a heavy chain, which are located within CDR3 of the 3005a heavy chain. Deamidation at these two asparagine residues within the 3005a heavy chain correlated with a decrease in the biological activity (e.g., T cell activation activity) of epcoritamab (see Example 1).

In an embodiment, the deamidated variant of epcoritamab comprises deamidation of the N333/N330 residues on the 3005a/3001d heavy chains. In an embodiment, the deamidated variant of epcoritamab comprises deamidation of the N397/N394 on the 3005a/3001d heavy chains.

In an embodiment, the oxidized variant of epcoritamab comprises oxidation at one or more tryptophan and/or methionine residues. In an embodiment, the oxidized species of epcoritamab comprises oxidation of the W53 residue on the 3001d heavy chain. In an embodiment, the oxidized species of epcoritamab comprises oxidation of the W93 residue on the 3005a light chain. In an embodiment, the oxidized species of epcoritamab comprises oxidation of the W94 residue on the 3001d light chain. The W53 (3001d HC), W93 (3005a LC) and W94 (3001d LC) residues are located within CDR regions and thus potentially could impact the biological activity of epcoritamab.

In an embodiment, the oxidized variant of epcoritamab comprises oxidation at M260/M257 residues on the 3005a/3001d heavy chains. In an embodiment, the oxidized variant of epcoritamab comprises oxidation of M436/M433 residues on the 3005a/3001d heavy chains.

Additional epcoritamab variants are described below and in the Examples.

### Highly Purified Compositions

In one aspect, the disclosure pertains to highly purified epcoritamab compositions. In an embodiment, the composition comprises epcoritamab, wherein the composition comprises at least 95% epcoritamab and less than 5% of an impurity (ies), e.g., a protein variant and/or an impurity. In other embodiments, the composition comprises less than 4.5%, 4.0%, 3.5%, 3.0%, 2.5%, 2.0%, 1.5%, 1.0%, 0.9%, 0.8%, 0.7%, 0.6%, 0.5%, 0.4%, 0.3%, 0.2%, 0.1%, 0.05% or 0.01% of a protein variant and/or an impurity (product-related impurity and/or process-related impurity). In other embodiments, the highly purified epcoritamab composition comprises epcoritimab (e.g., at least 95%) and between 0.0001%-5%, or 0.0001%-4%, or 0.0001%-3%, or 0.0001%-2%, or 0.0001% to 1% of an impurity (ies) e.g., a protein variant and/or an impurity. In other embodiments, the composition comprises epcoritamab (e.g., at least 95%) and between 0.001%-5%, or 0.001%-4%, or 0.001%-3%, or 0.001%-2%, or 0.001% to 1% of an impurity (ies). In other embodiments, the composition comprises epcoritamab (e.g., at least 95%) and between 0.01%-5%, or 0.01%-4%, or 0.01%-3%, or 0.01%-2%, or 0.01% to 1% of an impurity (ies).

In certain embodiments, the highly purified epcoritamab composition potentially comprises more than one impurity (e.g., two, three, four, five or more impurities, e.g., protein variants and/or product- or process-related impurities), wherein each unwanted component is present in the composition at a concentration of less than 5%, 4.5%, 4.0%, 3.5%, 3.0%, 2.5%, 2.0%, 1.5%, 1.0%, 0.9%, 0.8%, 0.7%, 0.6%, 0.5%, 0.4%, 0.3%, 0.2%, 0.1%, 0.05% or 0.01%. In other embodiments, each unwanted component is present in the composition at a concentration range between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1%.

In certain embodiments, the highly purified epcoritamab composition potentially comprises more than one impurity (e.g., two, three, four, five or more impurities, e.g., protein variants and/or product- or process-related impurities), wherein the total concentration of all unwanted components present in the composition is less than 5%, 4.5%, 4.0%, 3.5%, 3.0%, 2.5%, 2.0%, 1.5%, 1.0%, 0.9%, 0.8%, 0.7%, 0.6%, 0.5%, 0.4%, 0.3%, 0.2%, 0.1%, 0.05% or 0.01%. In other embodiments, the concentration range of the total of all unwanted components present in the composition is between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1%.

In some embodiments, the composition comprises less than 200-fold, 100-fold, 50-fold, 40-fold, 30-fold, 25-fold, 20-fold, 15-fold, 10-fold or 5-fold of the level of the Permitted Daily Exposure (PDE) for the impurity (ies), e.g., protein variant and/or product-related impurity and/or process-related impurity.

In some embodiments, the composition comprises less than 100 ug/ml, 90 ug/ml, 80 ug/ml, 70 ug/ml, 60 ug/ml, 50 ug/ml, 40 ug/ml, 30 ug/ml, 25 ug/ml, 20 ug/ml, 15 ug/ml, 10 ug/ml, 9 ug/ml, 8 ug/ml, 7 ug/ml, 6 ug/ml, 5 ug/ml, 4 ug/ml, 3 ug/ml, 2 ug/ml, 1 ug/ml, 0.9 ug/ml, 0.8 ug/ml, 0.7 ug/ml, 0.6 ug/ml, 0.5 ug/ml, 0.4 ug/ml, 0.3 ug/ml, 0.2 ug/ml, 0.1 ug/ml, 0.05 ug/ml, 0.01 ug/ml, 0.005 ug/ml or 0.001 ug/ml of an impurity (ies), e.g., a protein variant and/or product-related impurity and/or process-related impurity.

In some embodiments, the composition comprises between 0.0001-100 ug/ml, 0.0001-90 ug/ml, 0.0001-80 ug/ml, 0.0001-70 ug/ml, 0.0001-60 ug/ml, 0.0001-50 ug/ml, 0.0001-40 ug/ml, 0.0001-30 ug/ml, 0.0001-25 ug/ml, 0.0001-20 ug/ml, 0.0001-15 ug/ml, 0.0001-10 ug/ml, 0.0001-9 ug/ml, 0.0001-8 ug/ml, 0.0001-7 ug/ml, 0.0001-6 ug/ml, 0.0001-5 ug/ml, 0.0001-4 ug/ml, 0.0001-3 ug/ml, 0.0001-2 ug/ml, 0.0001-1 ug/ml, 0.0001-0.9 ug/ml, 0.0001-0.8 ug/ml, 0.0001-0.7 ug/ml, 0.0001-0.6 ug/ml, 0.0001-0.5 ug/ml, 0.0001-0.4 ug/ml, 0.0001-0.3 ug/ml, 0.0001-0.2 ug/ml, 0.0001-0.1 ug/ml, 0.0001-0.05 ug/ml, 0.0001-0.01 ug/ml, 0.0001-0.005 ug/ml or 0.0001-0.001 ug/ml of an impurity (ies), e.g., a protein variant and/or product-related impurity and/or process-related impurity.

In some embodiments, the composition comprises between 0.001-100 ug/ml, 0.001-90 ug/ml, 0.001-80 ug/ml, 0.001-70 ug/ml, 0.001-60 ug/ml, 0.001-50 ug/ml, 0.001-40 ug/ml, 0.001-30 ug/ml, 0.001-25 ug/ml, 0.001-20 ug/ml, 0.001-15 ug/ml, 0.001-10 ug/ml, 0.001-9 ug/ml, 0.001-8 ug/ml, 0.001-7 ug/ml, 0.001-6 ug/ml, 0.001-5 ug/ml, 0.001-4 ug/ml, 0.001-3 ug/ml, 0.001-2 ug/ml, 0.001-1 ug/ml, 0.001-0.9 ug/ml, 0.001-0.8 ug/ml, 0.001-0.7 ug/ml, 0.001-0.6 ug/ml, 0.001-0.5 ug/ml, 0.001-0.4 ug/ml, 0.001-0.3 ug/ml, 0.001-0.2 ug/ml, 0.001-0.1 ug/ml, 0.001-0.05 ug/ml or 0.001-0.01 ug/ml of an impurity (ies), e.g., a protein variant and/or product-related impurity and/or process-related impurity.

In some embodiments, the composition comprises between 0.01-100 ug/ml, 0.01-90 ug/ml, 0.01-80 ug/ml, 0.01-70 ug/ml, 0.01-60 ug/ml, 0.01-50 ug/ml, 0.01-40 ug/ml, 0.01-30 ug/ml, 0.01-25 ug/ml, 0.01-20 ug/ml, 0.01-15 ug/ml, 0.01-10 ug/ml, 0.01-9 ug/ml, 0.01-8 ug/ml, 0.01-7 ug/ml, 0.01-6 ug/ml, 0.01-5 ug/ml, 0.01-4 ug/ml, 0.01-3 ug/ml, 0.01-2 ug/ml, 0.01-1 ug/ml, 0.01-0.9 ug/ml, 0.01-0.8 ug/ml, 0.01-0.7 ug/ml, 0.01-0.6 ug/ml, 0.01-0.5 ug/ml, 0.01-0.4 ug/ml, 0.01-0.3 ug/ml, 0.01-0.2 ug/ml or 0.01-0.1 ug/ml of an impurity (ies), e.g., a protein variant and/or product-related impurity and/or process-related impurity.

For compositions comprising more than one impurity (e.g., two, three, four, five or more impurity), in an embodiment the upper limit concentration or concentration range of each impurity in ug/ml is set individually. In another embodiment, the upper limit concentration or concentration range in ug/ml is set for the total amount of impurities in the composition.

Principles to use for setting acceptance criteria for impurity levels are established in the art and are described in further detail in Example 2, Table 4. In some embodiments, the acceptance criteria are set using an approach selected from the group consisting of a published limit approach, a target limit approach, a stability limit approach and an empirical limit approach.

### Protein Variants and Product-Related Impurities

In some embodiments, an impurity is a protein variant or a product-related impurity, e.g., impurity relating to alteration of the epcoritamab structure. Evaluations of protein variants and product-related impurities are described in further detail in Example 4.

In some embodiments, the protein variant or product-related impurity is selected from the group consisting of a high molecular weight species (HMWS) of epcoritamab, a low molecular weight species (LMWS) of epcoritamab, a charge variant of epcoritamab, an acidic species of epcoritamab, a basic species of epcoritamab, a deamidated species of epcoritamab, a glycosylated species of epcoritamab, a glycated species of epcoritamab, an oxidized species of epcoritamab, a residual IgG1 homodimer intermediate, an N-terminal modification of epcoritamab, an aggregate of epcoritamab, a fragment of epcoritamab, a residual signal peptide modification of epcoritamab, an Fc fragment of epcoritamab and a Fab fragment of epcoritamab.

In an embodiment, the protein variant or product-related substance is high molecular weight species (HMWS) of epcoritamab. HMWS impurities of epcoritamab are described in further detail in Example 4. In some embodiments, the composition comprises less than 5.0%, 4.5%, 4.0%, 3.5%, 3.0%, 2.5%, 2.0%, 1.5%, 1.0% or 0.5% of an HMWS product-related substance, or between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1% of an HMWS product-related substance.

In an embodiment, the protein variant or product-related substance is low molecular weight species (LMWS) of epcoritamab. LMWS impurities of epcoritamab are described in further detail in Example 4. In some embodiments, the composition comprises less than 5.0%, 4.5%, 4.0%, 3.5%, 3.4%, 3.3%, 3.2%, 3.1%, 3.0%, 2.5%, 2.0%, 1.5%, 1.0% or 0.5% of an LMWS product-related substance, or between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1% of an LMWS product-related substance.

In an embodiment, the protein variant or product-related substance is a deamidated acidic species of epcoritamab. In some embodiments, the deamidated acidic species of epcoritamab comprises deamidation of residues selected from the group consisting of a N103 residue in the heavy chain of the CD3 binding arm of epcoritamab, a N106 residue in the heavy chain of the CD3 biding arm of epcoritamab, N103 and N106 residues in the heavy chain of the CD3 binding arm of epcoritamab, a N333 residue on the CD3 heavy chain of epcoritamab, a N330 residue in the heavy chain of the CD20 binding arm of epcoritamab, and combinations thereof. In some embodiments, the composition comprises less than 5.0%, 4.5%, 4.0%, 3.5%, 3.0%, 2.5%, 2.0%, 1.5%, 1.0% or 0.5% of a deamidated acidic species product-related substance, or between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1% of a deamidated acidic species of epcoritamab.

In an embodiment, the protein variant or product-related substance is a glycosylated species of epcoritamab. In some embodiments, the glycosylated species comprises glycosylation of residues selected from the group consisting of a N106 residue in the heavy chain of the CD3 binding arm of epcoritamab, a N305 residue in the heavy chain of the CD3 binding arm of epcoritamab, a N302 residue in the heavy chain of the CD20 binding arm of epcoritamab, and combinations thereof. In some embodiments, the composition comprises less than 5.0%, 4.5%, 4.0%, 3.5%, 3.0%, 2.5%, 2.0%, 1.5%, 1.0% or 0.5% of a glycosylated species product-related substance, or between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1% of a glycosylated species of epcoritamab.

In an embodiment, the protein variant or product-related substance is a glycated species of epcoritamab. In some embodiments, the glycated species comprises glycation of residues selected from the group consisting of a K331 residue in the heavy chain of the CD20 binding arm of epcoritamab, a K334 residue in the heavy chain of the CD3 binding arm of epcoritamab, and combinations thereof. In some embodiments, the composition comprises less than 5.0%, 4.5%, 4.0%, 3.5%, 3.0%, 2.5%, 2.0%, 1.5%, 1.0% or 0.5% of a glycated species product-related substance, or between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1% of a glycated species of epcoritamab.

In an embodiment, the protein variant or product-related substance is an oxidized species of epcoritamab. In some embodiments, the oxidized species comprises oxidation of residues selected from the group consisting of a W53 residue in the heavy chain of the CD20 binding arm of epcoritamab, a W93 residue in the light chain of the CD3 binding arm of epcoritamab, a W94 residue in the light chain of the CD20 binding arm of epcoritamab, a M363 residue in the heavy chain of the CD20 binding arm of epcoritamab, a M366 residue in the heavy chain of the CD3 bindgin arm of epcoritamab, M260/M257 residues of 3005a/3001d, M436/M433 residues of 3005a/3001d, and combinations thereof. In some embodiments, the composition comprises less than 5.0%, 4.5%, 4.0%, 3.5%, 3.0%, 2.5%, 2.0%, 1.5%, 1.0% or 0.5% of an oxidized species product-related substance, or between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1% of an oxidized species of epcoritamab.

In an embodiment, the protein variant or product-related substance is an N-terminal modification of epcoritamab. In some embodiments, the N-terminal modification is selected from the group consisting of a glutamate to pyroglutamate conversion, a residual signal peptide attached to epcoritamab, and combinations thereof. In some embodiments, the glutamate to pyroglutamate conversion occurs in at least one of a heavy chain of the CD3 binding arm of epcoritamab, a heavy chain of the CD20 binding arm of epcoritamab and a light chain of the CD20 binding arm of epcoritamab. In some embodiments, the glutamine to pyroglutamate conversion occurs in the light chain of the CD3 binding arm of epcoritamab. In some embodiments, the residual signal peptide comprises an amino acid sequence selected from the group consisting of (a) Ser-Glu-Ala and (b) Leu-Cys-Thr-Gly-Ser-Glu-Ala (SEQ ID NO: 9). In some embodiments, the composition comprises less than 5.0%, 4.5%, 4.0%, 3.5%, 3.0%, 2.5%, 2.0%, 1.5%, 1.0% or 0.5% of an N-terminal modification of epcoritamab, or between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1% of an N-terminal modification of epcoritamab.

### Process-Related Impurities

In other embodiments, an impurity is a process-related impurity or process-related substance. Evaluation of process-related impurities is described in further detail in Example 3.

In some embodiments, the process-related impurity is selected from the group consisting of a reducing agent, an oxidizing agent, a manufacturing intermediate, a residual host cell protein, a host cell nucleic acid, a media component and a chromatographic material.

In an embodiment, the process-related impurity is a reducing agent. In an embodiment, the reducing agent is 2-mercaptoethylamine (2-MEA). Evaluation of 2-MEA as an impurity is described in detail in Example 3. For example, a composition comprising 48 mg of epcoritamab was shown to have as little as 7.68 ug of 2-MEA remaining in the composition after purification, which is over 13-fold lower than the Permitted Daily Exposure (PDE) for 2-MEA (100 ug). In some embodiments, the reducing agent (e.g., 2-MEA) is present in the composition at a level at least 13-fold, 12-fold, 11-fold, 10 fold, 9-fold, 8-fold, 7-fold, 6-fold or 5-fold lower than the PDE for 2-MEA. In some embodiments, the composition comprises less than 1.0%, 0.9%, 0.8%, 0.7%, 0.6%, 0.5%, 0.4%, 0.3%, 0.2%, 0.1%, 0.05% or 0.01% of the reducing agent (e.g., 2-MEA), or between 0.0001%-1%, or 0.0001%-0.9%, or 0.0001%-0.8%, or 0.0001%-0.7%, or 0.0001% to 0.6%, or 0.0001%-0.5%, or 0.0001%-0.4%, or 0.0001%-0.3%, or 0.0001%-0.2%, or 0.0001% to 0.1%, or 0.0001%-0.001%, or 0.001%-1%, or 0.01% to 1% of the reducing agent (e.g., 2-MEA).

In an embodiment, the process-related impurity is an oxidizing agent. In an embodiment, the oxidizing agent is dehydro-L-Ascorbic Acid (dhAA). Evaluation of dhAA as an impurity is described in detail in Example 3. For example, a composition comprising 48 mg epcoritamab was shown to have as little as 1.6 mg of dhAA remaining in the composition after purification, which is over 200-fold lower than the Permitted Daily Exposure (PDE) for dhAA (320 mg). In some embodiments, the oxidizing agent (e.g., dhAA) is present in the composition at a level at least 200-fold, 150-fold, 100-fold, 75-fold, 50-fold, 25-fold, or 10-fold lower than the PDE for dhAA. In some embodiments, the composition comprises less than 5.0%, 4.5%, 4.0%, 3.5%, 3.0%, 2.5%, 2.0%, 1.5%, 1.0% or 0.5% of the oxidizing agent (e.g., dhAA), or between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1% of the oxidizing agent (e.g., dhAA).

In an embodiment, the process-related impurity is a manufacturing intermediate of epcoritamab. In an embodiment, the manufacturing intermediate is the CD3-binding arm of epcoritamab, such as the 3005a antibody or a portion thereof. In an embodiment, the manufacturing intermediate is the CD20-binding arm of epcoritamab, such as the 3001d antibody or a portion thereof. The 3005a and 3001d antibody intermediates are described in detail in Example 1 and evaluation of 3005a and 3001d as process-related impurities is described in detail in Example 3. In some embodiments, the composition comprises less than 5.0%, 4.5%, 4.0%, 3.5%, 3.0%, 2.5%, 2.0%, 1.5%, 1.0% or 0.5% of the manufacturing intermediate(s), e.g., 3005a antibody or a portion thereof and/or 3001d antibody or a portion thereof, or between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1% of the manufacturing intermediate(s), e.g., 3005a antibody or a portion thereof and/or 3001d antibody or a portion thereof.

In an embodiment, the process-related impurity is a residual IgG1 homodimer intermediate. In some embodiments, the composition comprises less than 5.0%, 4.5%, 4.0%, 3.5%, 3.0%, 2.5%, 2.0%, 1.5%, 1.0% or 0.5% of a residual IgG1 homodimer intermediate process-related impurity, or between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1% of the residual IgG1 homodimer intermediate.

In an embodiment, the process-related impurity comprises residual host cell proteins. In some embodiments, the composition comprises less than 5.0%, 4.5%, 4.0%, 3.5%, 3.0%, 2.5%, 2.0%, 1.5%, 1.0% or 0.5% of residual host cell protein process-related impurity, or between 0.0001%-5%, or 0.0001%-4%, or 0.0001%-3%, or 0.0001%-2%, or 0.0001% to 1%, or 0.001%-5%, or 0.001%-4%, or 0.001%-3%, or 0.001%-2%, or 0.001% to 1%, or 0.01%-5%, or 0.01%-4%, or 0.01%-3%, or 0.01%-2%, or 0.01% to 1% of residual host cell protein process-related impurity.

### Impurities—Other Embodiments

In an embodiment, a product-related impurity of the disclosure is a charge variant of epcoritamab comprising a post-translational modification (PTM). Non-limiting examples of charge variants comprising a PTM include N-term E1-SEA (3005a HC), N-term E1-LCTGSEA (3005a HC), N103-Deamidated (3005a HC), N106-Deamidated (3005a HC), N103/106-G2F (3005a HC), and N103/N106-AIF (3005a HC).

In an embodiment, a product-related impurity of the disclosure is a deamidated epcoritamab variant comprising deamidation at heavy chain positions N103 and N106 in the CDR3 of the 3005a heavy chain.

In an embodiment, a product-related impurity of the disclosure is an acidic variant of epcoritamab comprising deamidation at heavy chain positions N103 and N106 in the CDR3 of the 3005a heavy chain.

In an embodiment, a product-related impurity of the disclosure is a HMWS of epcoritamab. In embodiments, the HMWS is a dimer or oligomer of epcoritamab. In an embodiment, the HMWS is a dimer comprising the dominant glycoform (G1F/G2F). In an embodiment, the dimer has a mass of 286 kDa. In an embodiment, the HMWS comprises an epcoritamab variant comprising a signal peptide comprising the amino acid sequence SEA. In an embodiment, the HMWS comprises an epcoritamab dimer comprising a signal peptide comprising the amino acid sequence SEA. In an embodiment, the HMWS comprises an epcoritamab variant comprising a signal peptide comprising the amino acid sequence LCTGSEA (SEQ ID NO: 9). In an embodiment, the HMWS comprises an epcoritamab dimer comprising a signal peptide comprising the amino acid sequence LCTGSEA (SEQ ID NO: 9).

In an embodiment, a product-related impurity of the disclosure is a LMWS of epcoritamab. In an embodiment, the LMWS is an Fc+Fab fragment of epcoritamab. In an embodiment, the Fc+Fab fragment of epcoritamab has a mass of 102 kDa. In an embodiment, the LMWS is HL and/or HHL, wherein H=heavy chain and L-light chain.

In an embodiment, a process-related impurity of the disclosure is a residual host cell protein (HCP), such as a residual HCP(s) remaining after recombinant expression of 3005a and 3001d. In an embodiment, the residual HCP is polyubiquitin. In embodiments, the residual HCP can be, for example, polyubiquitin, ubiquitin, IgG-binding protein A, peroxiredoxin-1, thioredoxin reductase 1 (cytoplasmic), phospholipase B-like, and/or combinations thereof.

In an embodiment, an impurity of the disclosure comprises a residual signal peptide. In an embodiment, the residual signal peptide comprises the amino acid sequence SEA. In an embodiment, the residual signal peptide comprises the amino acid sequence LCTGSEA (SEQ ID NO: 9).

In an embodiment, an impurity of the disclosure comprises a non-consensus glycosylation, such as GF2 or A1F. In an embodiment, the impurity comprising a non-consensus glycosylation is N103/106-G2F (3005a HC). In an embodiment, the impurity comprising a non-consensus glycosylation is N103/N106-A1F (3005a HC).

In an embodiment, an impurity of the disclosure comprises a deamidated species. In embodiments, deamidation occurs at one or more positions selected from 3005a heavy chain positions N103, 3005a heavy chain position N106, N397/N394 (3005a/3001d) and N333/N330 (3005a/3001d).

In an embodiment, an impurity of the disclosure comprises an oxidized species. In embodiments, an increase in oxidation occurs at positions selected from M260/M257 (3005a/3001d) and M436/M433 (3005a/3001d).

In embodiments, any one or more of the afore-mentioned impurities can be minimized in an epcoritamab composition, in accordance with the disclosure herein. In embodiments, the composition comprises less than 4.5%, 4.0%, 3.5%, 3.0%, 2.5%, 2.0%, 1.5%, 1.0%, 0.9%, 0.8%, 0.7%, 0.6%, 0.5%, 0.4%, 0.3%, 0.2%, 0.1%, 0.05% or 0.01% of the impurity. In other embodiments, the composition comprises epcoritimab (e.g., at least 95%) and between 0.0001%-5%, or 0.0001%-4%, or 0.0001%-3%, or 0.0001%-2%, or 0.0001% to 1% of the impurity. In other embodiments, the composition comprises epcoritamab (e.g., at least 95%) and between 0.001%-5%, or 0.001%-4%, or 0.001%-3%, or 0.001%-2%, or 0.001% to 1% of the impurity. In other embodiments, the composition comprises epcoritamab (e.g., at least 95%) and between 0.01%-5%, or 0.01%-4%, or 0.01%-3%, or 0.01%-2%, or 0.01% to 1% of the impurity.

In embodiments, the composition comprises less than 200-fold, 100-fold, 50-fold, 40-fold, 30-fold, 25-fold, 20-fold, 15-fold, 10-fold or 5-fold of the level of the PDE for the impurity.

In embodiments, the composition comprises less than 100 ug/ml, 90 ug/ml, 80 ug/ml, 70 ug/ml, 60 ug/ml, 50 ug/ml, 40 ug/ml, 30 ug/ml, 25 ug/ml, 20 ug/ml, 15 ug/ml, 10 ug/ml, 9 ug/ml, 8 ug/ml, 7 ug/ml, 6 ug/ml, 5 ug/ml, 4 ug/ml, 3 ug/ml, 2 ug/ml, 1 ug/ml, 0.9 ug/ml, 0.8 ug/ml, 0.7 ug/ml, 0.6 ug/ml, 0.5 ug/ml, 0.4 ug/ml, 0.3 ug/ml, 0.2 ug/ml, 0.1 ug/ml, 0.05 ug/ml, 0.01 ug/ml, 0.005 ug/ml or 0.001 ug/ml of the impurity.

In embodiments, the composition comprises between 0.0001-100 ug/ml, 0.0001-90 ug/ml, 0.0001-80 ug/ml, 0.0001-70 ug/ml, 0.0001-60 ug/ml, 0.0001-50 ug/ml, 0.0001-40 ug/ml, 0.0001-30 ug/ml, 0.0001-25 ug/ml, 0.0001-20 ug/ml, 0.0001-15 ug/ml, 0.0001-10 ug/ml, 0.0001-9 ug/ml, 0.0001-8 ug/ml, 0.0001-7 ug/ml, 0.0001-6 ug/ml, 0.0001-5 ug/ml, 0.0001-4 ug/ml, 0.0001-3 ug/ml, 0.0001-2 ug/ml, 0.0001-1 ug/ml, 0.0001-0.9 ug/ml, 0.0001-0.8 ug/ml, 0.0001-0.7 ug/ml, 0.0001-0.6 ug/ml, 0.0001-0.5 ug/ml, 0.0001-0.4 ug/ml, 0.0001-0.3 ug/ml, 0.0001-0.2 ug/ml, 0.0001-0.1 ug/ml, 0.0001-0.05 ug/ml, 0.0001-0.01 ug/ml, 0.0001-0.005 ug/ml or 0.0001-0.001 ug/ml of the impurity.

### Assays for Purity Evaluations

Methods for assessing the purity of a composition of the disclosure are well-established in the art and can be selected by the ordinarily skilled artisan based on the type of impurities to be evaluated. Non-limiting examples of suitable assays are described further below.

### Size Exclusion High Performance Liquid Chromatography

In an embodiment, epcoritamab composition is evaluated for impurities by assessing size heterogeneity within the composition. In an embodiment, size heterogeneity is assessed under native conditions by size exclusion high performance liquid chromatography (SE-HPLC). SE-HPLC can be used to determine the purity of the epcoritamab drug substance in a composition. The relative amounts of Monomer and high molecular weight substances (HMWS) can be determined. The analytical procedure was validated for specificity, repeatability, intermediate precision, reproducibility, limit of quantification, linearity, accuracy and range. Robustness of the analytical procedure was also evaluated. Further, SE-HPLC is suitable to quantitatively determine degradation products (HMWS) as a stability indicating assay.

The determination of Monomer and HMWS attributes provides a quantitative measurement of the size heterogeneity profile of an epcoritamab composition, for example, at the time of release from purification and upon shelf storage. In an embodiment, the acceptance criteria for Monomer and HMWS were: (i) equal to or greater than 97.2% monomer at release and equal to or greater than 97% monomer under long term storage condition; and (ii) equal to or less than 2.8% HMWS at release and equal to or less than 3.0% HMWS under long term storage conditions.

### Capillary Electrophoresis Sodium Dodecyl Sulfate (CE-SDS)

In an embodiment, an epcoritamab composition is evaluated for impurities by assessing mass heterogeneity within the composition. In an embodiment, mass heterogeneity is assessed under denaturing conditions by capillary electrophoresis sodium dodecyl sulfate (CE-SDS). In an embodiment, mass heterogeneity is assessed by CE-SDS under non-reducing conditions (CE-SDS, non-reduced), for example, to evaluate intact IgG and LMWS. In an embodiment, mass heterogeneity is assessed by CE-SDS under reducing conditions (CE-SDS, reduced), for example, to evaluate IgG as heavy chain (HC) and light chain (LC).

Acceptance criteria were set for Intact IgG, LMWS and IgG as HC and LC. In an embodiment, the acceptance criteria for Intact IgG and LMWS using CE-SDS (non-reduced) were: (i) equal to or greater than 94.2% Intact IgG at release and equal to or greater than 93.5% Intact IgG under long term storage condition; and (ii) equal to or less than 4.8% LMWS at release and equal to or less than 5.6% LMWS under long term storage conditions. In another embodiment, the acceptance criteria for IgG as HC and LC using CE-SDS (reduced) were equal to or greater than 94.2% IgG as HC and LC at release and equal to or greater than 93.8% IgG as HC and LC under long-term storage conditions.

Imaged Capillary Isoelectric Focusing (icIEF)

In an embodiment, an epcoritamab composition is evaluated for impurities by assessing charge heterogeneity within the composition. In an embodiment, charge heterogeneity is assessed using the imaged capillary isoelectric focusing (icIEF) method to resolve charged variants by their isoelectric points into Acidic, Neutral and Basic forms. Charged variants are separated according to their pI in the presence of an ampholytic pH gradient formed by an applied electric field across the capillary. Charge heterogeneity is quantitatively determined by the relative percentage area of each form. The analytical procedure was validated for specificity, repeatability, intermediate precision, reproducibility, linearity, accuracy and range. Robustness of the analytical procedure was also evaluated. Further, icIEF is suitable to quantitatively determine the charge form distribution as a stability indicating assay.

Acceptance criteria were set for Acidic, Neutral and Basic forms. In an embodiment, the acceptance criteria for Acidic forms using icIEF were: 57.2%-77.3% Acidic forms at release and 57.2%-79.5% Acidic forms under long term storage condition. In an embodiment, the acceptance criteria for Neutral forms using icIEF were: 20.8%-41.1% Neutral forms at release and 18.7%-41.1% Neutral forms under long term storage conditions. In an embodiment, the acceptance criteria for Basic forms using icIEF were: equal to or less than 3.4% Basic forms at release and equal to or less than 3.9% Basic forms under long term storage conditions.

### Cation Exchange High Performance Liquid Chromatography (CIEX-HPLC)

In an embodiment, an epcoritamab composition is evaluated for impurities by assessing residual biological intermediates within the composition, such as charged variants. In an embodiment, residual biological intermediates such as charged variants are assessed using cation exchange high performance liquid chromatography (CIEX-HPLC). The CIEX-HPLC method separates molecules according to their surface charge. Peaks relating to epcoritamab and biological intermediates (e.g., 3001d and 3005a) can be integrated. Acceptance criteria were set for Residual 3005a and Residual 3001d, with the upper limit for both being set to ≤5.0%.

### Liquid Chromatography-Mass Spectrometry (LC-MS) Peptide Mapping

The amino acid sequence of epcoritamab and protein variants thereof can be assessed by standard LC-MS peptide mapping. An epcoritamab composition can be digested by either trypsin or chymotrypsin under denaturing and reducing conditions. Resulting peptides are then subsequently separated using a reversed phase column followed by MS detection. The peptides are identified by comparing accurate mass and the fragmentation spectra produced by high energy collisional dissociation (HCD) and electron-transfer/higher-energy collision dissociation (EThcD) to an in-silico digestion of the epcoritamab amino acid sequence.

### Primary T Cell Activation and T Cell Cytotoxicity Assay

The ability of epcoritamab to induce activation of CD4-positive and CD8-positive T-cells, as well as T-cell mediated cytotoxicity, can be evaluated in a primary T-cell activation and cytotoxicity assay. The assay is based on multi-color flow cytometry whereby target cell viability of Daudi cells and T-cell activation of peripheral blood mononuclear cells is measured simultaneously in a co-culture system. In brief, PBMCs, a dilution series of epcoritamab reference standard or test sample and cultured Daudi cells are added into a 96-wells tissue culture plate and incubated. After incubation, the cells are washed, detection antibody-mix with fluorescently labeled markers is added and further incubated. Cells are washed and resuspended with acquisition buffer and analyzed by cell sorting flow cytometer. The fluorescent signal from viable B cells, as well as activated CD4⁺ and CD8⁺ T cells, is plotted against the concentration.

### Additional Assays

Additional assays can be used to evaluate the properties of the epcoritamab compositions of the disclosure.

In an embodiment, the biological activity of the composition is evaluated. In an embodiment, the biological activity is evaluated by a T cell activation bioassay. For example, the biological activity of an epcoritamab composition can be determined in a surrogate T-cell activation assay using GloResponse™ NFAT-Luc2 Jurkat cells expressing CD3 and Daudi cells expressing CD20. Co-binding of the BsAb to both the Daudi and to the GloResponse NFAT-Luc2 Jurkat cells activates the NFAT (nuclear factor of activated T-cells) pathway subsequent to luciferase expression. The bioluminescent signal is detected using luciferase substrate Bio-Glo™ and by means of a multimode reader. The biological activity of epcoritamab can be reported as % relative potency relative to a reference standard. The acceptance criterion was established based on the mean±3SD. In an embodiment, the acceptance criteria for the T cell activation bioassay for an epcoritamab composition is 65%-135%.

In an embodiment, protein concentration of the composition is evaluated. In an embodiment, protein concentration is evaluated by UV detection. Protein concentration can be determined by measuring absorbance at 280 nm (A280) using a variable light path instrument. By measuring the absorbance of a sample at various pathlengths, a linear relationship between path length and absorbance can be determined. The protein concentration can be calculated from the slope of the linear regression line and the extinction coefficient. In an embodiment, the acceptance criteria is set at target concentration±10%. In an embodiment, the acceptance criteria is 60.0 mg/mL±6.0 mg/mL.

The pH of the composition can be evaluated, for example, by potentiometry. The pH of the composition typically is determined by the pH of the formulation buffer, which is controlled by weighing of buffering components during buffer preparation. In an embodiment, acceptance criteria for pH is set at a range from pH 5.3 to 5.7. In an embodiment, acceptance criteria for pH is set at pH 5.5±0.3.

Bacterial endotoxins within the composition can be evaluated, for example by the chromogenic kinetic method. The approach used to set the acceptance criterion was based on the maximum tolerable bacterial endotoxins for subcutaneous dosing described in the pharmacopeia. The bacterial endotoxins limit provides an approximate safety margin of 2-fold within the maximum endotoxins limit of 5 EU/kg body weight for parenteral drugs defined in USP. In an embodiment, acceptance criteria for bacterial endotoxins is set at ≤1.0 EU/mg protein.

The bioburden of the composition can be evaluated, for example by standard membrane filtration. In an embodiment, acceptance criteria for bioburden is set at ≤10 CFU/100 mL

### Preparation and Purification Methods

Highly purified epcoritamab compositions of the disclosure can be prepared according to the preparation and purification methods described herein. Methods of preparing and purifying the epcoritamab compositions of the disclosure are described in detail in Examples 1 and 2. To prepare epcoritamab, the CD3 and CD20 binding arms are first prepared and then assembled into the epcoritamab bispecific antibody, followed by processes as described herein to remove unwanted components from the resultant composition.

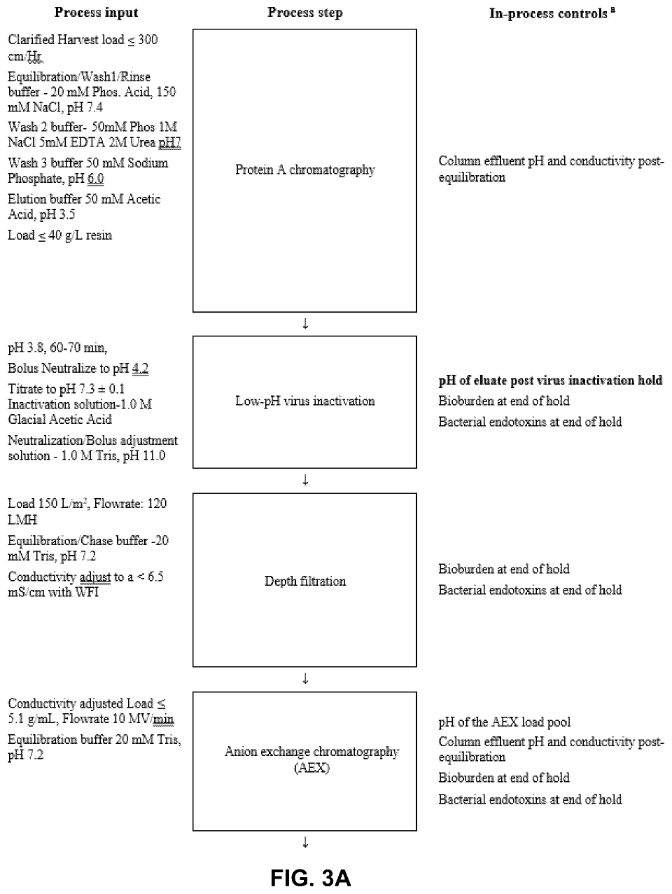

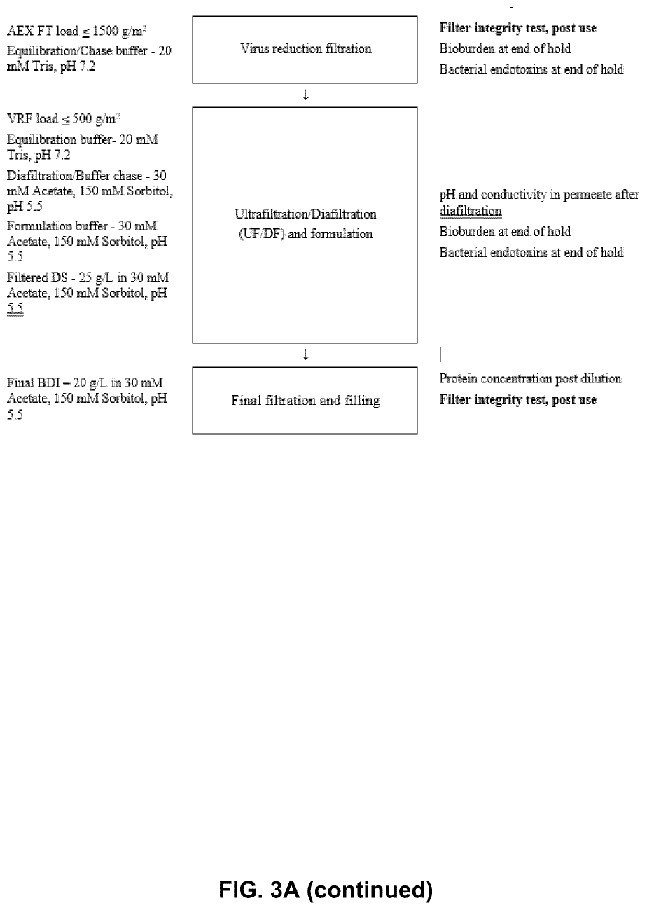

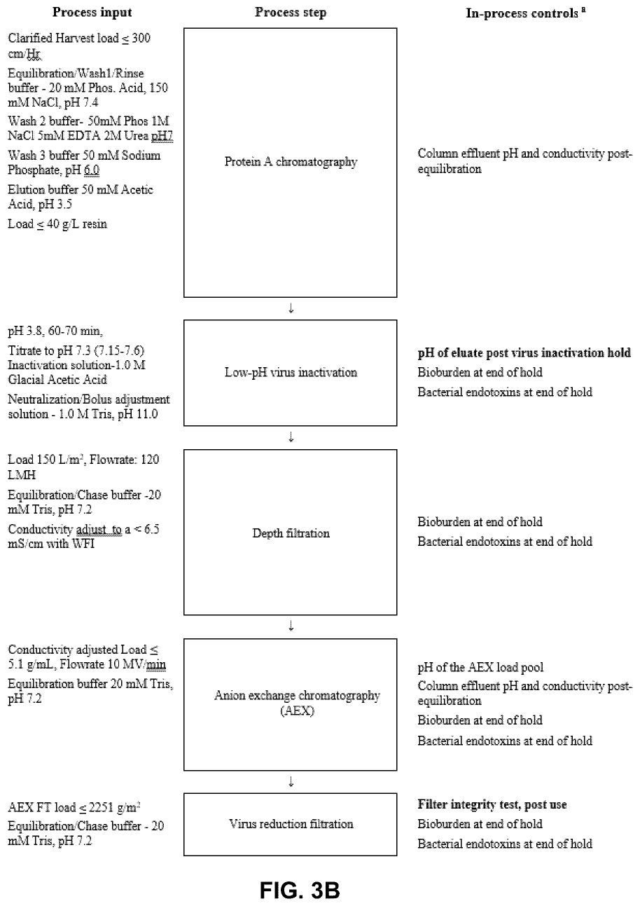

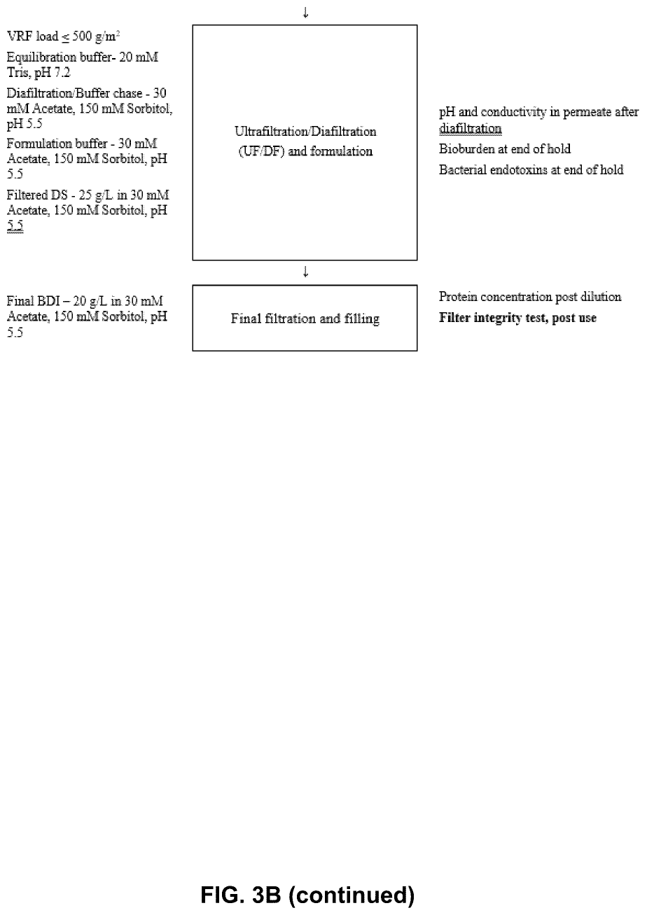

Non-limiting examples of preparation and purification process workflows for the CD3 and CD20 binding arms of epcoritamab are illustrated schematically in FIG. 3A and FIG. 3B, respectively. Preparation and purification of the CD3 and CD20 binding arms is also described further in U.S. Pat. No. 10,544,220; US Patent Publication 2017/0355767; and PCT Publication WO 2016/110576. In brief, the binding arms are expressed recombinantly in host cells (e.g., CHO cells) in a bioreactor process. Following harvest, the binding arms can be purified by applying, for example, protein A chromatography, low pH virus inactivation, depth filtration, anion exchange chromatography, virus reduction filtration, ultrafiltration/diafiltration (UF/DF), and final filtration and filling. In an embodiment, a CD3 binding arm (3005a) is purified according to the conditions (e.g., loads, flowrates, buffer components, buffer pH) as set forth in FIG. 3A. In an embodiment, a CD20 binding arm (3001d) is purified according to the conditions (e.g., loads, flowrates, buffer components, buffer pH) as set forth in FIG. 3B.

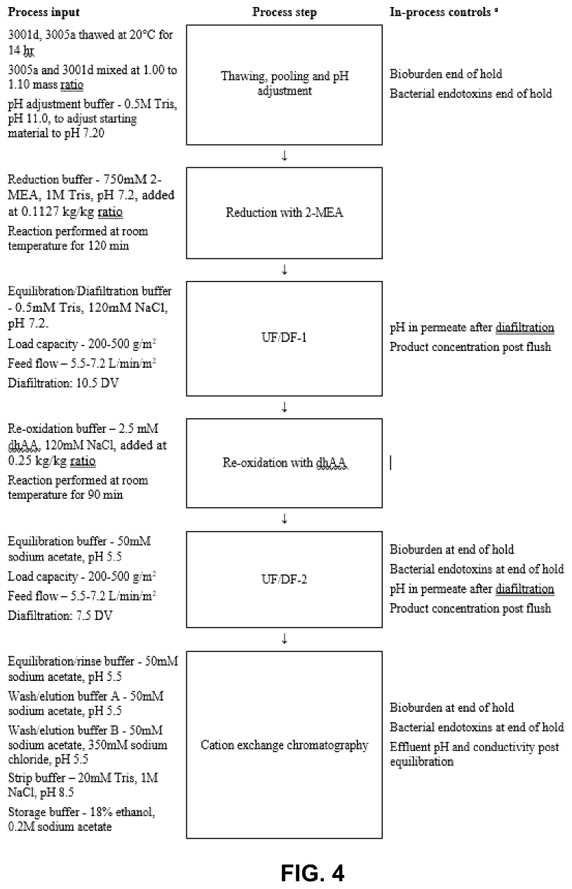

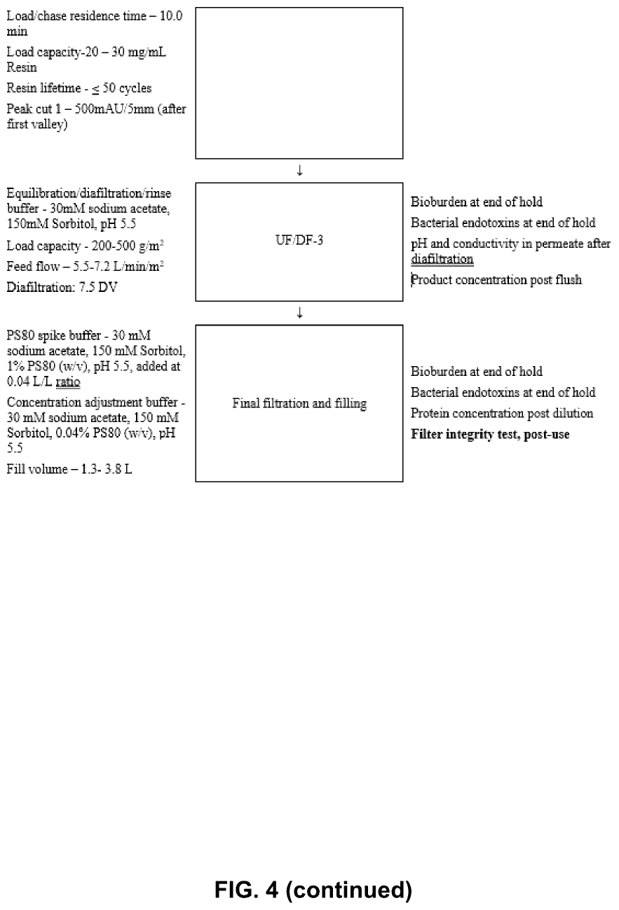

A non-limiting example of a preparation and purification process workflow for the epcoritamab bispecific antibody is illustrated schematically in FIG. 4. Accordingly, in embodiments, an epcoritamab composition is purified according to the processes and conditions (e.g., loads, flowrates, buffer components, buffer pH) as set forth in FIG. 4.

In an embodiment, the method comprises combining a sample of the first binding arm of the epcoritamab (3005a antibody) and a sample of the second binding arm of epcoritamab (3001d antibody), to thereby form a combined sample, followed by exposing the combined sample to reducing conditions to form a reduced combined sample. The reducing conditions facilitate the conjugation of the first and second binding arms. In an embodiment, the reducing conditions comprise exposure to a reducing agent. In an embodiment, the reducing agent is 2-MEA.

Following reduction, the reduced combined sample is treated with an oxidizing agent to form an oxidized sample. In an embodiment, the oxidizing agent is dhAA.

Following oxidation, the oxidized sample is subjected to cation exchange chromatography to purify and isolate the now highly-purified bispecific antibody composition.

Accordingly, in one aspect, the disclosure provides a method of preparing an epcoritamab composition of the disclosure, epcoritamab being comprised of a first binding arm and a second binding arm, the method comprising:
(a) combining a first sample of the first binding arm and a second sample of the second binding arm to form a combined sample and exposing the combined sample to reducing conditions to form a reduced combined sample;
(b) treating the reduced combined sample with an oxidizing agent to form an oxidized sample; and
(c) subjecting the oxidized sample to cation exchange chromatography such that the composition is prepared.

The starting batch scale can be, for example, 1-5 kg of material (e.g., 3 kg). Reduction can be performed at, for example, 18-31° C., e.g., at 18-25° C. (room temperature). The eluate from the cation exchange chromatography step can be obtained, for example, using an elution buffer comprising 50 mM sodium acetate, 350 mM sodium chloride, pH 5.5.

In some embodiments, the samples of the first binding arm and the second binding arm are pooled to form the combined sample and the pH of the combined sample is adjusted, e.g., to pH 7.0-7.7, e.g., 7.1-7.3. The pH can be adjusted by standard methods, e.g., with 0.5 M Tris.

Accordingly, in another embodiment, the method comprises:
(a) combining a first sample of the first binding arm and a second sample of the second binding arm to form a combined sample;
(b) adjusting the pH of the combined sample to pH 7.0-7.7;
(c) exposing the combined sample to reducing conditions to form a reduced combined sample;
(d) treating the reduced combined sample with an oxidizing agent to form an oxidized sample; and
(e) subjecting the oxidized sample to cation exchange chromatography such that the composition is prepared.

In other embodiments, the samples are subjected to ultrafiltration/diafiltration (UF/DF) between each of the various steps of the method. Appropriate UF/DF conditions can be determined by the ordinarily skilled artisan. For example, loads of 150-550 g/m² can be used (e.g., 500 g/m²) and room temperature (18-25° C.). A non-limiting examples of diafiltration buffers include 50 mM sodium acetate, pH 5.5. Alternatively, 20 mM sodium phosphate, pH 7.5, can be used as a diafiltration buffer.

Accordingly, in another embodiment, the method comprises:
(a) combining a first sample of the first binding arm and a second sample of the second binding arm to form a combined sample (e.g., with pH adjusted to 7.0-7.7) and exposing the combined sample to reducing conditions to form a reduced combined sample;
(b) performing UF/DF on the reduced combined sample;
(c) treating the reduced combined sample with an oxidizing agent to form an oxidized sample;
(d) performing UF/DF on the oxidized sample;
(e) subjecting the oxidized sample to cation exchange chromatography to obtain an eluate; and
(f) performing UF/DF on the eluate such that the composition is prepared.

In other embodiments, a final filtration step is performed after step (f) to thereby prepare the highly purified composition.

### Pharmaceutical Formulations

The highly purified epcoritamab compositions of the disclosure can be further formulated, for example into pharmaceutical formulations. Pharmaceutical formulations comprising epcoritamab have been described in the art (e.g., US Patent Publications 2021/0032358 and 2022/0411505; PCT Publications WO 2019/155008 and WO 2021/028587). In certain embodiments, the pharmaceutical formulation comprises epcoritamab, acetate, sorbitol and a surfactant. In an embodiment, epcoritamab is present in the formulation at a concentration in a range of 0.5 to 120 mg/ml (e.g., 1-100 mg/ml, 10-60 mg/ml, 60 mg/ml, 48 mg/ml, 24 mg/ml, 10 mg/ml, 5 mg/ml). In an embodiment, the acetate is present in the formulation at concentration in a range of 20-40 mM (e.g., 30 mM). In an embodiment, sorbitol is present in the formulation at a concentration in a range of 140 to 260 mM (e.g., 150-250 mM, 150 mM, 250 mM). In an embodiment, the surfactant is a polysorbate, e.g., polysorbate 20 or polysorbate 80. In an embodiment, the surfactant is present in the formulation at a concentration in a concentration range of 0.005% to 0.4% (e.g., 0.01%, 0.4%).

In some embodiments, the pharmaceutical formulation is a stable liquid composition suitable for subcutaneous administration.

In some embodiments, the pharmaceutical formulation is a lyophilized composition that is reconstituted and suitable for subcutaneous administration.

In some embodiments, the pharmaceutical formulation is a stable liquid composition suitable for subcutaneous administration. In one embodiment, the pharmaceutical formulation is not subject to prior lyophilization and/or reconstitution. In one embodiment, the pharmaceutical formulation suitable for subcutaneous administration is a sterile, preservative-free, clear to slightly opalescent, colorless to slightly yellow solution, practically free of visible particles.

In some embodiments, the composition comprises about 4 to 5 mg/mL of epcoritamab. In some embodiments, the composition comprises about 60 mg/mL of epcoritamab.

In one embodiment that pharmaceutical composition is administered by intravenous or subcutaneous injection or infusion.

### Uses

The highly purified epcoritamab compositions of the disclosure can be used in any circumstance in which a CD3×CD20 bispecific antibody would be of benefit, such as in the treatment of cancers including B cell malignancies. Use of CD3×CD20 BsAbs in the treatment of cancers has been described in the art, for example in the treatment of chronic lymphocytic leukemia (CLL) (see e.g., US Patent Publication 2022/0112287; PCT Publication WO 2022/053653), follicular lymphoma (see e.g., U.S. Pat. Nos. 11,535,679; 11,608,383; PCT Publication WO 2022/053655; PCT Publication WO 2022/053656), and diffuse large B cell lymphoma (DLBCL) (see e.g., U.S. Pat. No. 11,548,952; PCT Publication WO 2022/053654; PCT Publication WO 2022/053657; PCT Publication WO 2022/053658).

The epcoritamab compositions of the disclosure also have additional utility in therapy and diagnosis of a variety of CD20-related diseases. For example, the composition can be used to elicit in vivo or in vitro one or more of the following biological activities: to inhibit the growth of and/or differentiation of a cell expressing CD20; to kill a cell expressing CD20; to mediate phagocytosis or ADCC of a cell expressing CD20 in the presence of human effector cells; to mediate CDC of a cell expressing CD20 in the presence of complement; to mediate apoptosis of a cell expressing CD20; and/or to induce translocation into lipid rafts upon binding CD20.

In another embodiment, the compositions of the disclosure can be used to effect T cell-mediated immune responses, inflammation and microenvironment re-modelling.

In a particular embodiment, the compositions are used in vivo to treat, prevent or diagnose a variety of CD20-related diseases. Examples of CD20-related diseases include, among others, B cell lymphoma, e.g., large B cell lymphoma (e.g., diffuse LBCL), non-Hodgkin's lymphoma (NHL), B cell leukemia and immune diseases, e.g., autoimmune diseases, such as those listed below.

In one embodiment, the compositions of the disclosure are used for the treatment of large B cell lymphoma (LBCL), such as diffuse LBCL.

In one embodiment, the compositions of the disclosure are used for the treatment of NHL or B cell leukemia.

In one embodiment, the compositions of the disclosure are used for the treatment of CD20 antibody-resistant NHL or B cell leukemia, such as rituximab- or ofatumumab-resistant NHL or B cell leukemia, e.g. rituximab-resistant non-aggressive B-cell lymphoma.

In one embodiment, the compositions of the disclosure are used for the treatment of Acute Lymphoblastic Leukemia (ALL), such as relapsed or refractory ALL.

In one embodiment, the compositions of the disclosure are used for the treatment of CLL, such as relapsed or refractory CLL.

In one embodiment, the compositions of the disclosure are used for the treatment of FL, such as or relapsed or refractory FL.

The Examples in this specification are not intended to, and should not be used to, limit the invention; they are provided only to illustrate the invention.

### Example 1: CD3×CD20 Bispecific Antibody Epcoritamab and Protein Variants Thereof

Bispecific antibodies targeting CD3 and CD20, as well as recombinant production thereof, are described in detail in U.S. Pat. No. 10,544,220; US Patent Publication 2017/0355767; and PCT Publication WO 2016/110576, the entire contents of each of which are specifically incorporated herein by reference. In this example, the structure of epcoritamab is described, along with certain undesired protein variants thereof that were identified through forced degradation of the bispecific antibody.

### Epcoritamab Structure and Function

Epcoritamab is a CD3×CD20 bispecific antibody composed of two heavy chains and two light chains. The heavy chains belong to the gamma (γ) isotype, form 1 (G1), and the light chains to the kappa (κ) and lambda (λ) type, making epcoritamab a bispecific IgG1λκ antibody. The two heavy chains are bound to each other by two interchain disulfide bonds, and one light chain is bound to one heavy chain by a single interchain disulfide bond. Each light chain has two intrachain disulfide bonds, and each heavy chain has four intrachain disulfide bonds. The structure of epcoritamab is shown schematically in FIG. 1. Epcoritamab carries inertness mutations to silence Fc-mediated effector functions. It has characteristics typical of a human IgG1antibody, including normal neonatal Fc receptor (FcRn) binding and in vivo stability. The bispecific antibody molecule has properties inherited from each of the parental antibodies. Epcoritamab simultaneously binds to CD3 on T-cells and CD20 on malignant B-cells, inducing CD20-specific T-cell activation and T-cell-mediated cytotoxicity.

### Structures of 3005a and 3001d Intermediates

Epcoritamab is generated using the DuoBody® technology by a process called controlled Fab-arm exchange. The two parental antibodies 3005a and 3001d are shown schematically in FIG. 2A and FIG. 2B, respectively. Each contain a matched point mutation (F405L and K409R, respectively), facilitating the formation of the DuoBody® molecule, epcoritamab. 3005a and 3001d are each monomers in solution.

The 3005a antibody (CD3 binding arm) is a glycoprotein belonging to the immunoglobulin (Ig) superfamily, composed of two heavy chains and two light chains. The heavy chains belong to the gamma (γ) isotype, form 1 (G1), and the light chains to the lambda (λ) type, making 3005a an IgG1λ antibody. The two heavy chains are bound to each other by two interchain disulfide bonds, and one light chain is attached to one heavy chain by a single interchain disulfide bond. Each light chain has two intrachain disulfide bonds, and the heavy chains has four intrachain disulfide bonds. The amino acid sequences for the heavy and light chains of 3005a are shown below in Table 1.

TABLE 1
Amino acid sequence for 3005a
Heavy Chain (454 aa)(SEQ ID NO: 1)
EVKLVESGGG LVQPGGSLRL SCAASGFTFN TYAMNWVRQA PGKGLEWVAR IRSKYNNYAT YYADSVKDRF
TISRDDSKSS LYLQMNNLKT EDTAMYYCVR HGNFGNSYVS WFAYWGOGTL VTVSSASTKG PSVFPLAPSS
KSTSGGTAAL GCLVKDYFPE PVTVSWNSGA LTSGVHTFPA VLQSSGLYSL SSVVTVPSSS LGTQTYICNV
NHKPSNTKVD KRVEPKSCDK THTCPPCPAP EFEGGPSVFL FPPKPKDTLM ISRTPEVTCV VVAVSHEDPE
VKFNWYVDGV EVHNAKTKPR EEQYNSTYRV VSVLTVLHQD WINGKEYKCK VSNKALPAPI EKTISKAKGQ
PREPQVYTLP PSREEMTKNQ VSLTCLVKGF YPSDIAVEWE SNGQPENNYK TTPPVLDSDG SFLLYSKLTV
DKSRWQQGNV FSCSVMHEAL HNHYTQKSLS LSPG
Light Chain (215 aa) (SEQ ID NO: 2)
QAVVTQEPSF SVSPGGTVTL TCRSSTGAVT TSNYANWVQQ TPGQAFRGLI GGTNKRAPGV PARFSGSLIG
DKAALTITGA QADDESIYFC ALWYSNLWVF GGGTKLTVLG QPKAAPSVTL FPPSSEELQA NKATLVCLIS
DFYPGAVTVA WKADSSPVKA GVETTTPSKQ SNNKYAASSY LSLTPEQWKS HRSYSCOVTH EGSTVEKTVA
PTECS
The four mutated amino acids in the heavy chain are L234F, L235E, D265A and F405L, using Eu numbering.
These positions are marked in bold.
Complementarity-determining regions (CDR) are underlined in the sequence.

Three-point mutations L234F, L235E and D265A were introduced in the Fc domain to silence Fc-mediated effector functions. The F405L point mutation was introduced to facilitate formation of the epcoritamab DuoBody®. The amino acids are numbered according to Eu numbering. Using direct numbering, the point mutations appear as amino acid number 242, 243, 273 and 413.

The 3001d antibody (CD20 binding arm) is a glycoprotein belonging to the immunoglobulin (Ig) superfamily, composed of two heavy chains and two light chains. The heavy chains belong to the gamma (γ) isotype, form 1 (G1), and the light chains to the kappa (κ) type, making 3001d an IgG1κ antibody. The two heavy chains are bound to each other by two interchain disulfide bonds, and one light chain is attached to one heavy chain by a single interchain disulfide bond. Each light chain has two intrachain disulfide bonds, and the heavy chains has four intrachain disulfide bonds. The amino acid sequences for the heavy and light chains of 3001d are shown below in Table 2.

TABLE 2
Amino Acid Sequence of 3001d
Heavy Chain (451 aa)(SEQ ID NO: 3)
EVQLVESGGG LVQPDRSLRL SCAASGFTFH DYAMHWVRQA PGKGLEWVST ISWNSGTIGY ADSVKGRFTI
SRDNAKNSLY LQMNSLRAED TALYYCAKDI QYGNYYYGMD VWGQGTTVTV SSASTKGPSV FPLAPSSKST
SGGTAALGCL VKDYFPEPVT VSWNSGALTS GVHTFPAVLQ SSGLYSLSSV VTVPSSSLGT QTYICNVNHK
PSNTKVDKRV EPKSCDKTHT CPPCPAPEFE GGPSVFLFPP KPKDTLMISR TPEVTCVVVA VSHEDPEVKE
NWYVDGVEVH NAKTKPREEQ YNSTYRVVSV LTVLHQDWLN GKEYKCKVSN KALPAPIEKT ISKAKGQPRE
PQVYTLPPSR EEMTKNQVSL TCLVKGFYPS DIAVEWESNG QPENNYKTTP PVLDSDGSFF LYSRLTVDKS
RWQQGNVFSC SVMHEALHNH YTQKSLSLSP G
Light Chain (214 aa) (SEQ ID NO: 4)
EIVLTQSPAT LSLSPGERAT LSCRASQSVS SYLAWYQQKP GQAPRLLIYD ASNRATGIPA RFSGSGSGTD
FTLTISSLEP EDFAVYYCQQ RSNWPITFGQ GTRLEIKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSEN
RGEC
The four mutated amino acids in the heavy chain are L234F, L235E, D265A and K409R, using Eu numbering.
These positions are marked in bold.
Complementarity-determining regions (CDR) are underlined in the sequence.

Three-point mutations L234F, L235E and D265A were introduced in the Fc domain to silence Fc-mediated effector functions. The K409R point mutation was introduced to facilitate formation of the epcoritamab DuoBody®. The amino acids are numbered according to Eu numbering. Using direct numbering, the point mutations appear as amino acid number 239, 240, 270 and 414.

### Epcoritamab Variants Identified by Forced Degradation Experiments

Potential degradation pathways of epcoritamab were assessed under various forced degradation conditions, including thermal, photo, oxidation, low pH, high pH, glycation and freeze/thaw stress. These studies allowed for the identification of epcoritamab protein variants that could potentially interfere with the biological activity of epcoritamab. Forced degradation samples were incubated over time (e.g., 4 weeks) and were characterized (e.g., weekly) by LC-MS (reduced peptide mapping), icIEF (charge heterogeneity), CIEX-HPLC (charge heterogeneity), SE-HPLC (size heterogeneity), reduced CE-SDS (denaturing mass heterogeneity), non-reduced CE-SDS (denaturing mass heterogeneity). Samples also were evaluated in the T-cell activation bioassay described below to determine biological activity. Based on these studies, epcoritamab product quality impact was predominantly observed in four major categories: aggregation, fragmentation, oxidation and charge heterogeneity.

### Primary T-Cell Activation and T-Cell Mediated Cytotoxicity Assay

The ability of epcoritamab to induce activation of CD4-positive and CD8-positive T-cells, as well as T-cell mediated cytotoxicity, was evaluated in a primary T-cell activation and cytotoxicity assay. The assay is based on multi-color flow cytometry whereby target cell viability of Daudi cells and T-cell activation of peripheral blood mononuclear cells is measured simultaneously in a co-culture system. In brief, PBMCs, a dilution series of epcoritamab reference standard or test sample and cultured Daudi cells were added into a 96-wells tissue culture plate and incubated. After incubation, the cells were washed, detection antibody-mix with fluorescently labeled markers was added and further incubated. Cells were washed and resuspended with acquisition buffer and analyzed by cell sorting flow cytometer. The fluorescent signal from viable B cells as well as activated CD4⁺ and CD8⁺ T cells were plotted against the concentration. The dose response curves were analyzed using 4-parameter logistic model fitting and the percentage activity relative to reference standard was reported.

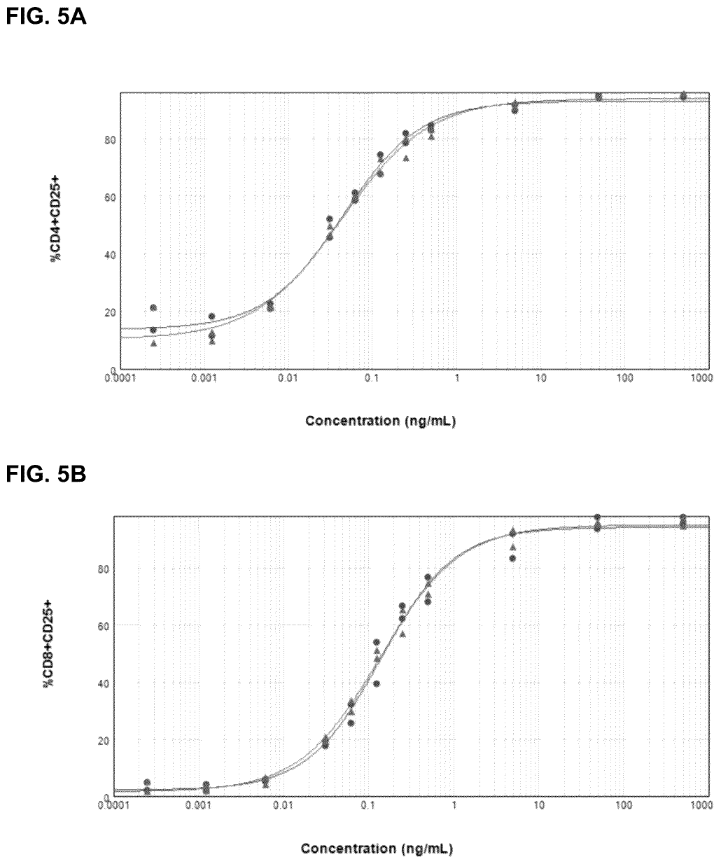

Representative epcoritamab dose response curves for activation of CD4⁺ CD25+ T cells and CD8⁺ CD25+ T cells are shown in FIG. 5A and FIG. 5B, respectively. Epcoritamab shows a dose-dependent increase of CD4- and CD8-positive T cell activation. The mean percentage of T cell activation based upon three independent determinations was found to be 87% for CD4+ T cells and 95% for CD8+ T cells for epcoritamab compared to a reference control.

### Acidic Species and Effect on Biological Activity of Epcoritamab

Under thermal stress, an increase in Acidic forms of approximately 17% was observed in the icIEF and CIEX methods after 8 weeks of incubation at 40° C. This observation correlated with the LC-MS peptide mapping analysis, which identified increased deamidation at heavy chain positions N103 (3005a), N106 (3005a) and N333/N330 (3005a/3001d). The T-cell activation assay revealed that samples incubated at 40° C. for up to 8 weeks had biological activity comparable to the reference, indicating that this level of Acidic forms under thermal stress did not significantly affect epcoritamab activity.

Under high pH, a significant increase in Acidic forms was observed after 4 weeks of incubation at pH 9.0 in the icIEF and CIEX methods, with increases of approximately 27% and 55%, respectively. This observation correlated with the LC-MS peptide mapping analysis which identified increased deamidation primarily at heavy chain positions N103 (3005a), N106 (3005a), N397/N394 (3005a/3001d) and N333/N330 (3005a/3001d). The LC-MS data also revealed a minor increase of oxidation at M260/M257 (3005a/3001d) and M436/M433 (3005a/3001d).

N103 and N106 in the CDR3 of 3005a heavy chain showed the greatest level of deamidation increase to approximately 50% and 60% deamidation, respectively. As the deamidation level of these two asparagine residues significantly increased, the biological activity of the 4-week sample in the T cell activation assay showed a significant decrease (from 97% at time 0 to 32% at 4 weeks), indicating a correlation between the deamidation of these two asparagine residues in CDR3 of 3005a heavy chain and the biological activity of epcoritamab. The extent of loss in biological activity increased with a higher deamidation level on these two sites under high pH conditions as compared to the heat stressed samples. While high pH conditions also led to relatively low level of increase in HMWS and LMWS species, the major contribution to biological activity loss was considered to be due to the deamidation of N103 and N106 (3005a). Thus, this forced degradation study identified protein variants of epcoritamab having deamidation at heavy chain positions N103 and N106 in the CDR3 of 3005a heavy chain as product-related impurities (Acidic forms) that are of particular interest to minimize in an epcoritamab composition of the disclosure.

### Oxidized Species

The main oxidized species of epcoritamab identified by forced degradation resulted from oxidation of the W53 residue on the 3001d heavy chain, M260/M257 residues on the 3005a/3001d heavy chains, M436/M433 residues on the 3005a/3001d heavy chains, W93 residue on the 3005a light chain and W94 residue on the 3001d light chain. The photo stress-induced oxidation of W53, W93 and W94 yielded different oxidation products identified as mono-oxidation, di-oxidation (N-formyl kynurenine) and kynurenine as previously described (Li et al., 2014). Residues W53 (3001d HC), W94 (3001d LC) and W93 (3005a LC) are located in CDR regions and can therefore potentially impact the biological activity, which correlates with the observed decrease of T-cell activation activity for the photo-stressed samples.

### Example 2: Manufacturing Process for CD3×CD20 Bispecific Antibody Epcoritamab

To prepare epcoritamab, the CD3 and CD20 binding arms (3005a and 3001d) are first prepared and then combined to prepare the bispecific antibody. Non-limiting examples of preparation and purification process workflows for the CD3 (3005a) and CD20 (3001d) binding arms of epcoritamab are illustrated schematically in FIG. 3A and FIG. 3B, respectively. In brief, the binding arms are expressed recombinantly in host cells (e.g., CHO cells) in a bioreactor process. Following harvest, the binding arms can be purified by applying, for example, protein A chromatography, low pH virus inactivation, depth filtration, anion exchange chromatography, virus reduction filtration, ultrafiltration/diafiltration (UF/DF), and final filtration and filling. Non-limiting examples of conditions for carrying out these processes (e.g., loads, flowrates, buffer components and pHs, and the like) include those set forth in FIG. 3A and FIG. 3B.

A flow diagram of the epcoritamab drug substance (DS) manufacturing process, including buffers and in-process controls, is shown in FIG. 4. Accordingly, non-limiting examples of conditions for carrying out these processes (e.g., loads, flowrates, buffer components and pHs, and the like) include those set forth in FIG. 4. The epcoritamab DS manufacturing process starts by thawing and pooling the biological intermediates, 3001d and 3005a, followed by a controlled reduction process using 2-mercaptoethylamine hydrochloride (2-MEA). The material is then ultra/diafiltrated (UF/DF-1) to remove the reducing agent, and Dehydro-L-Ascorbic Acid (dhAA) is added to the retentate to allow for active re-oxidation and reformation of the disulfide bonds. The material is ultra/diafiltrated (UF/DF-2) to remove residual dhAA prior to cation exchange chromatography followed by an ultra/diafiltration (UF/DF-3) to concentrate the protein. Polysorbate 80 (PS80) is then added and the protein concentration is adjusted prior to filling into bags and freezing. All steps are performed at ambient temperature (18° C. to 25° C.), unless specified otherwise. All UF/DF membranes are single use.

The scale of the manufacturing process was defined by the combined amounts of biological intermediates 3001d and 3005a that goes into each batch of epcoritamab drug substance. The batch scale was 3 kg (±0.3 kg). For one epcoritamab DS batch, multiple batches of the individual BIs, 3001d and 3005a, were used. All BI batches used for epcoritamab DS manufacturing complied with the acceptance criteria for release.

Certain buffer attributes (e.g., pH, conductivity) studied during process characterization were found to impact defined performance parameters. Additionally, the UF/DF-3 buffer was monitored due to its known impact on epcoritamab DS formulation. These attributes were actively controlled at buffer release and, if necessary, buffers are rejected prior to use in manufacturing. The buffer attributes which impacted performance parameters and their acceptable ranges are summarized below in Table 3.

TABLE 3
Buffer attributes identified to impact performance
parameters of epcoritamab DS process

| | | | Accept- |
|---|---|---|---|
| | Buffer or solution | | able |
| Process step | designation | Attribute | range |
| Cation | Equilibration/rinse/chase | pH | 5.4-5.6 |
| exchange | buffer; Wash/elution buffer | | |
| chromatography | A buffer | | |
| | Wash/elution buffer B buffer | pH | 5.4-5.6 |
| UF/DF-3 | Equilibration/DF buffer/ | pH | 5.3-5.7 |
| | recovery UF/DF-3 | Conductivity | 1.0-3.0 |
| | | (mS/cm) | |

### Acceptance Criteria

Selection of quality attributes and appropriate acceptance criteria for release and stability testing ensure the suitability of drug substance for further processing to safe and efficacious epcoritamab drug product.

Acceptance criteria for epcoritamab DS were calculated according to the principles set forth in Table 4 using available release and stability data of batches representative of the commercial product/process. These calculations together with considerations of multiple non-statistical factors were used to set the specifications for epcoritamab DS.

TABLE 4
Principles used for setting acceptance criteria

| Principles | Brief description |
|---|---|
| Published limit approach | Applies to quality attributes for which acceptable limits have been established and |
| | published in Pharmacopeia, regulatory guidance, or described in relevant scientific |
| | literature. |
| | This approach is used for Identity, Bacterial endotoxins, and Bioburden. |
| Target limit approach | Used for attributes that are defined by the design or formulation of the drug |
| | substance and/or drug product and for which no change over time is expected. |
| | Acceptance criteria are set around a pre-determined target based on manufacturing |
| | capability to assure product consistency. |
| | This approach is used for Opalescence, Color, Protein concentration, Polysorbate 80 |
| | and pH. |
| Stability limit approach | For attributes that may change during shelf life, acceptance criteria are based on |
| | prediction of process performance, analytical variability and shelf life estimation |
| | using available stability data. |
| | This approach is used for mass, charge, and size heterogeneity. |
| Empirical limit approach | This approach applies to attributes for which the acceptance criteria cannot be set by |
| | one of the other approaches due to inherent method variability. Acceptance criteria |
| | are set based on manufacturing knowledge. This approach also applies to attributes |
| | reported as threshold limits or below a method quantitation limit. |
| | This approach is used for Biological activity and Residual biological intermediates. |

### Control of Critical Steps and Intermediates

A comprehensive control strategy was established for the epcoritamab drug substance (DS) by identifying critical quality attributes (CQAs). Process characterization has identified that certain process steps can impact CQAs of the product. The control of these critical steps is important for the control of the CQAs. A critical step is defined as a process step that contains critical process parameters (CPPs) or critical in-process controls (IPCs). The identified critical steps include Thawing, Pooling and pH adjustment of 3005a and 3001d; Cation Exchange Chromatography; UF/DF-3 and Final Filtration and Filling. The CPPs and critical IPCs are components of the drug substance control strategy, which are shown below in Table 5 and 6, respectively.

TABLE 5
Critical process parameters

| Critical step | CPP | Acceptable range | CQAs known to be impacted |
|---|---|---|---|
| Thawing, pooling | Biological | 1.00:1.045 to | Residual biological intermediates |
| and pH adjustment | intermediate | 1.00:1.250 g:g | |
| of 3005a and 3001d | mixing ratio | | |
| | 3005a:3001d | | |
| Cation Exchange | Load capacity | 18-32 mg/mL_{Resin} | Charge related variants (Basic |
| Chromatography | | | forms) |
| UF/DF-3 | DF-Factor | 6.0-9.0 | pH |
| Final Filtration and | Addition factor of | 0.038 to | Polysorbate content |
| Filling | PS80 spike buffer | 0.042 L/L | |
| Final Filtration and | Target protein | 58.0 to | Protein concentration |
| Filling | concentration after | 64.0 mg/mL | |
| | concentration | | |
| | adjustment | | |

TABLE 6
Critical in-process controls

| | | Acceptance | CQAs known to |
|---|---|---|---|
| Critical step | Critical IPC | criteria | be impacted |
| Final | Filter integrity test, | Pass | Microbial |
| filtration | post-use (bubble point | | contaminants |
| and filling | test) | | |

### Process Validation and Evaluation

The set points and acceptable ranges for process parameters were set based on development, process characterization knowledge, and historical experience with the DuoBody® platform process. The process validation strategy was designed to demonstrate that the commercial process can deliver epcoritamab DS of consistent and acceptable quality to support further manufacture of drug product.

All validation and acceptance criteria were met for the four consecutive validation batches demonstrating process consistency during Thawing, Pooling of 3005a and 3001d, reduction with 2-mercaptoethylamine hydrochloride (2-MEA), UF/DF-1, re-oxidation with Dehydro-L-Ascorbic Acid (dhAA), UF/DF-2, cation exchange chromatography, UF/DF-3 as well as final filtration and filling. Validation of the manufacturing process for epcoritamab DS was successfully completed. Consistent results for process parameters and performance parameters demonstrate the drug substance process can be controlled effectively and performs reproducibly to yield drug substance that meets the expected quality.

As described in further detail in Examples 2 and 3, it was demonstrated that process-related and product-related impurities were well controlled in the epcoritamab DS manufacturing process described herein. In conclusion, the epcoritamab DS manufacturing process has been successfully qualified.

### Example 3: Assessment of Process-Related Impurities

Potential process-related impurities in epcoritamab drug substance (DS) were assessed or monitored during the manufacturing process to ensure that the process results in acceptable impurity levels. Process-related impurities were those derived from the manufacturing process that is described in Example 2 and set forth schematically in FIG. 4. These include impurities from the epcoritamab DS manufacturing process or from raw materials used in the process. The process-related impurities in the biological intermediates 3001d and 3005a (such as residual host cell DNA, residual host cell protein (HCP) or residual Protein A that could be carried into the epcoritamab DS) are controlled at the 3001d and 3005a manufacturing stage.

### Residual Host Cell Protein Analysis

For analysis of residual host cell proteins remaining after recombinant expression of 3005a and 3001d, all samples were analyzed both with and without standards. The seven standards used were: myoglobin, beta-lactoglobulin, lysozyme C, hemoglobin subunit alpha, hemoglobin subunit beta, alpha-lactalbumin and serotransferrin. For samples with standards, a protein standard mixture of the seven intact proteins was spiked into the samples to be used as internal protein standards for calculation of median response factor and for quality control. The seven standards were spiked-in at approximately 2000 ng/mg sample protein (˜2000 ppm) into 100 ug for the harvest samples and 200 ug for the remaining samples. The sample volumes were equalized by adding NH4HCO3 and the proteins were precipitated in four volumes acetone over night at −20° C.

For enzymatic digestion, the precipitated proteins were dissolved in 8M urea, reduced with dithiothreitol and carbamidomethylated using iodoacetamide. The proteins were digested by lysyl endopeptidase followed by trypsin, and the resulting peptides desalted and concentrated before LC-MS/MS analysis.

Protein identification was determined by IDA-LC-MS/MS. Samples were analyzed in triplicate on an Exigent nanoLC connected online to a TripleTOF 6600 mass spectrometer (Sciex). The peptides (4 μg for harvest samples, 8 ug for purification steps) were separated on a reversed phase C18 CSH-column using a 40 min gradient at a flow of 5 μL/min. The data was acquired in data-dependent mode where a survey spectrum (m/z range 350-1700) is followed by MS/MS (m/z range 130-2000) of the most intense multiply charged ions using collision induced dissociation.

Protein quantification was determined by SWATCH-LC-MS/MS analysis. For SWATH analysis the peptides were separated using the same chromatography as the IDA analysis. The SWATH analyses consist of a parent ion spectrum, followed by 46 MS/MS acquisitions that transmit and fragment all ions within variable window sizes from m/z 350-1106. All samples were analyzed in triplicate by SWATH-LC-MS/MS.

For protein identification by sequence, the IDA-LC-MS/MS data were searched against a protein sequence database using Protein Pilot software (Sciex) to create sample specific protein libraries. The ion libraries with identified proteins and peptides were imported into Peak View (Sciex) for protein quantification. All proteins were imported including shared peptides. For quantification, the SWATH data were compared with the ion libraries using the following processing settings: Max 1000 peptides per protein, 6 transitions per peptide, 95% Peptide Confidence Threshold, 1% False Discovery Rate Threshold, modified peptides were excluded, 2 min XIC extraction window, 75 ppm XIC width.

The resulting protein and peptide areas were exported to Excel for data analysis and were quantified using the SumALL algorithm.

Every protein has its own unique MS-response factor, i.e., MS signal per ng protein, in the specific sample matrix and digest conditions. The quantification of the HCPs was based on the median response factor calculated for the seven internal standard proteins digested and processed under the same conditions. The amount of the individual standard protein was calculated using the median response factor and compared to the known amount spiked into the sample. The accuracy between the actual and measured protein amount was calculated for QC purposes. Five to seven of the seven standard proteins were generally within 50% to 200% of the expected, corresponding to +/−2-fold. Proteins outside of this range represent a minor part of all proteins (incl. HCPs) that either have very high or very low response factors in the given sample matrix. The amount of the individual HCPs was also calculated from the median response factor.

The criteria for HCP identification and for quantification was as follows. Proteins were identified by minimum 2 peptides and quantified above 50 ppm in the harvest sample and above 25 ppm in the remaining samples (25/50 ng/mg sample protein, general LLOQ of the assay). HCP identifications by 2 peptides and below LLOQ are included, but with uncertain quantification. The coefficient of variation (CV, standard deviation/average) was used to find protein quantifications requiring manual evaluation: CV>0.3, above LLOQ. The quantification of a few proteins for each sample was modified manually. In all cases the quantification was modified due to a wrong assignment of a peptide spectrum by the software.

The HCP profile for the CD3 binding arm 3005a prepared according to the representative process set forth in FIG. 3A (Process 2) as compared to a related but slightly different process (Process 1) is shown below in Table 7:

TABLE 7
HCP profile for 3005a prepared by Process 1 or Process 2

| | | | | | ELISA | |
|---|---|---|---|---|---|---|
| | Number of HCPs | | | | Release | Specific HCPs |
| Process | Harvest | ProA | Sarto | Bulk | Result | (LC-MS/MS) |
| Process | 790 | 86 | 6 | 5 | 26.0 | ubiquitin, IgG-binding |
| 1 | | | | | ng/mg | protein A, |
| | | | | | | peroxiredoxin-1, |
| | | | | | | thioredoxin reductase 1 |
| | | | | | | (cytoplasmic), AGA |
| Process | 407 | 6 | 2 | 1 | <3.0 | Ubiquitin/ |
| 2 | | | | | ng/mg | polyubiquitin |

The HCP profile for the CD20 binding arm 3001d prepared according to the representative process set forth in FIG. 3B (Process 2) as compared to a related but slightly different process (Process 1) is shown below in Table 8:

TABLE 8
HCP profile for 3001d prepared by Process 1 or Process 2

| | | | | | ELISA | |
|---|---|---|---|---|---|---|
| | Number of HCPs | | | | Release | Specific HCPs |
| Process | Harvest | ProA | Sarto | Bulk | Result | (LC-MS/MS) |
| Process | 828 | 68 | 4 | 3 | 14.0 | ubiquitin, IgG-binding |
| 1 | | | | | ng/mg | protein A, |
| | | | | | | phospholipase B-like |
| Process | 392 | 4 | 1 | 1 | <3.0 | Ubiquitin/ |
| 2 | | | | | ng/mg | polyubiquitin |

The level of HCP generated using Process 2 was also quantification by LC-MS, the results of which showed that 46 ppm is the highest level of HCP measured across 8 batches prepared according to Process 2 (FIG. 3A and FIG. 3B).

### 2-MEA (Reducing Agent)

The reducing agent 2-mercaptoethylamine (2-MEA) is used for reduction of 3001d and 3005a. The amount of 2-MEA in samples was tested using a validated RP-HPLC method. Based on the targeted 2-MEA addition and assuming no process clearance, the maximal 2-MEA content in epcoritamab DS was calculated to be 463 ug/mg.

The primary function of the UF/DF1 step is removal of residual 2-MEA. However, 2-MEA was also monitored at the UF/DF2 step to further confirm full clearance. Analytical results for 2-MEA levels (in ug/mg) after the UF/DF1 and UF/DF2 steps and in the final epcoritamab DS are shown below in Table 9.

TABLE 9
Analytical Results for 2-MEA

| Step | 2-MEA results (μg/mg) | | | | | | | |
|---|---|---|---|---|---|---|---|---|
| UF/DF | <0.08 ^{b} | 0.10 | <0.08 ^{b} | <0.16 ^{a} | <0.16 ^{a} | <0.16 ^{a} | <0.16 ^{a} | <0.16 ^{a} |
| 1 | | | | | | | | |
| UF/DF | NT | NT | NT | <0.16 ^{a} | <0.16 ^{a} | <0.16 ^{a} | <0.16 ^{a} | <0.16 ^{a} |
| 2 | | | | | | | | |
| DS | <0.08 ^{b} | <0.08 ^{b} | <0.08 ^{b} | <0.08 ^{b} | <0.08 ^{b} | <0.08 ^{b} | <0.08 ^{b} | <0.08 ^{b} |

NT: Not tested.
^{a} Below LOQ of IPC.
^{b} Below LOQ of DS.

Thus, the results demonstrate robust and consistent removal of the reducing agent 2-MEA for all batches. The results from UF/DF1, UF/DF2, and at epcoritamab DS release, demonstrated that the level of 2-MEA is consistent throughout the batches produced with the commercial process, including the PPQ batches. All 2-MEA levels after UF/DF1 were below 0.16 ug/mg. Therefore, a 48 mg dose of epcoritamab drug product (DP) would contain less than 7.68 ug 2-MEA, which is 13-fold lower than the PDE of 100 μg 2-MEA per day.

Thus, assessment of the amount of residual reducing agent 2-MEA after the manufacturing process demonstrated that the level of this process-related impurity was found not to be of concern.

dhAA (Oxidizing Agent)

The oxidizing agent dhAA is used during the re-oxidation step of the controlled Fab-arm exchange to re-oxidize 3001d and 3005a to form the epcoritamab DS. The amount of dhAA was not tested due to the low amount added. Therefore, a risk-based assessment was performed.

The potential risk posed to patient safety from residual dhAA present in the epcoritamab DS process was calculated. The calculation assumes no clearance in the process after adding dhAA. With this assumption, it is calculated that a 48 mg dose of epcoritamab DP would contain 1.6 mg dhAA, which is 200-fold lower than the PDE of 320 mg dhAA per day. The worst-case calculation is presented in Table 10 below.

TABLE 10
Worst case calculation for dhAA

| | | Parameter in |
|---|---|---|
| Parameter | Number | calculation |
| Minimum batch size (g) | 2700 | A |
| Lowest observed overall drug substance process yield (%) | 48.5 | B |
| Minimum amount of epcoritamab based on lowest observed yield (g) | 1310 | C = A × B/100 |
| Highest amount of dhAA in buffer for addition (50 L) (g) | 43.99 | D |
| Maximum dhAA concentration in UF/DF2 retentate pool after dhAA | 0.03358 | E = D/C |
| addition relative to epcoritamab content when accounting for lowest | | |
| observed yield (mg/mg) | | |
| Maximum daily dose epcoritamab (mg/dose) | 48 | F |
| dhAA amount per dose (mg/dose) | 1.6 | G = E × F |

Thus, assessment of the amount of residual oxidizing agent dhAA after the manufacturing process demonstrated that the level of this process-related impurity was found not to be of concern.

Residual 3001d and/or 3005a Intermediates

The residual 3001d and/or 3005a intermediates are process-related impurities from the Fab-arm exchange which have not been cleared by the subsequent purification process. The amount of residual biological intermediates was monitored at release of epcoritamab DS using a validated CIEX HPLC method. Analytical results for the residual biological intermediates after cation exchange and in the epcoritamab DS are shown below in Table 11.

TABLE 11
Analytical results for residual biological intermediates

| Step | | Residual 3001d results (%) | | | | | | | |
|---|---|---|---|---|---|---|---|---|---|
| Cation | Cycle | NA ^{b} | NA ^{b} | NA ^{b} | 0.3 | 0.3 | 0.3 | 0.2 | 0.2 |
| exchange | 1 | | | | | | | | |
| eluate | Cycle | NA ^{b} | NA ^{b} | NA ^{b} | 0.3 | 0.3 | 0.3 | 0.2 | 0.2 |
| | 2 | | | | | | | | |

| | | | | | | | | |
|---|---|---|---|---|---|---|---|---|
| DS | 0.6 | 1.0 | 0.4 | 0.3 | 0.4 | 0.3 | 0.3 | 0.3 |

Residual 3005a results (%)

| | | | | | | | | | |
|---|---|---|---|---|---|---|---|---|---|
| Cation | Cycle | NA ^{b} | NA ^{b} | NA ^{b} | <0.8 ^{a} | <0.8 ^{a} | <0.8 ^{a} | 1.0 | 1.2 |
| exchange | 1 | | | | | | | | |
| eluate | Cycle | NA ^{b} | NA ^{b} | NA ^{b} | <0.8 ^{a} | <0.8 ^{a} | 0.8 | 1.0 | 1.1 |
| | 2 | | | | | | | | |

| | | | | | | | | |
|---|---|---|---|---|---|---|---|---|
| DS | <0.8 ^{a} | <0.8 ^{a} | <0.8 ^{a} | 0.8 | <0.8 ^{a} | 0.9 | 1.0 | 1.1 |

NA: Not applicable
^{a} Below LOQ.
^{b} Results are not representative since a protein stabilizing step was not implemented at time of the batch.

Additional analytical results for the residual biological intermediates after the indicated stages of purification are shown below in Table 12.

TABLE 12
Analytical results for residual biological intermediates

| | | | | |
|---|---|---|---|---|
| Time Point (Hours) | 0 | 24 | 72 | 144 |

Chemical stability of the UF/DF-2 in-process pool stored at 23-27° C.

| | | | | |
|---|---|---|---|---|
| Residual 3005a (%) | 1.8 | 1.7 | 1.7 | 1.8 |
| Residual 3001d (%) | 8.8 | 8.8 | 8.8 | 8.8 |

Chemical stability of the cation exchange chromatography in-process pool stored at 23-27° C.

| | | | | |
|---|---|---|---|---|
| Residual 3005a (%) | <0.8 | 1.0 | 1.6 | 1.4 |
| Residual 3001d (%) | 0.3 | 0.3 | 0.3 | 0.3 |

Chemical stability of the UF/DF-3 in-process pool stored at 23-27° C.

| | | | | | |
|---|---|---|---|---|---|
| Residual 3005a (%) | <0.8 | <0.8 | <0.8 | (96 hrs) | N/A |
| Residual 3001d (%) | 0.4 | 0.4 | 0.4 | (96 hrs) | N/A |

Chemical stability of the final filtration - epcoritamab DS pool stored at 23-27° C.

| | | | | | | |
|---|---|---|---|---|---|---|
| Residual 3005a (%) | <0.8 | <0.8 | <0.8 | (48 hrs) | <0.8 | (96 hrs) |
| Residual 3001d (%) | 0.4 | 0.4 | 0.4 | (48 hrs) | 0.4 | (96 hrs) |

As shown in Tables 11 and 12, analytical results of the various stages of epcoritamab DS purification demonstrate that the level of residual biological intermediates is low (1.2% or less) and consistent among all the DS batches, including the PPQ batches, indicating that the process is well controlled and robust.

### Example 4: Assessment of Product-Related Impurities

Potential product-related impurities in epcoritamab drug substance were assessed or monitored during development to ensure that the manufacturing process described in Example 2 (and illustrated schematically in FIG. 4) results in acceptable impurity levels.

Product-related impurities are molecular variants which may not have properties comparable to those of the desired product with respect to safety and efficacy, non-limiting examples of which include size variants such as high molecular weight species (HMWS) and low molecular weight species (LMWS), as well as charge variants such as acidic variants and basic variants.

### SE-HPLC Fractionation and Characterization of Size Variants

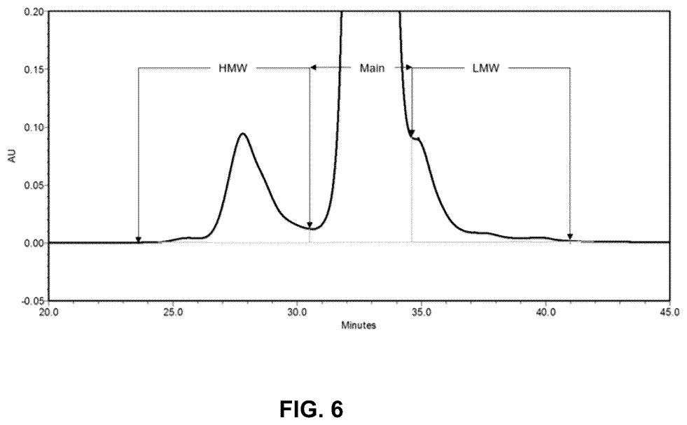

To isolate epcoritamab size variants in relatively high purity and sufficient quantities to investigate structure-function relationships, a highly resolving semi-preparative SE-HPLC method utilizing multiple columns was employed. FIG. 6 shows a representative chromatogram of a single injection. Subsequent characterization of the fractionated size variants established their identification and biological activity. Results for the HMWS, monomer, and LMWS are presented below.

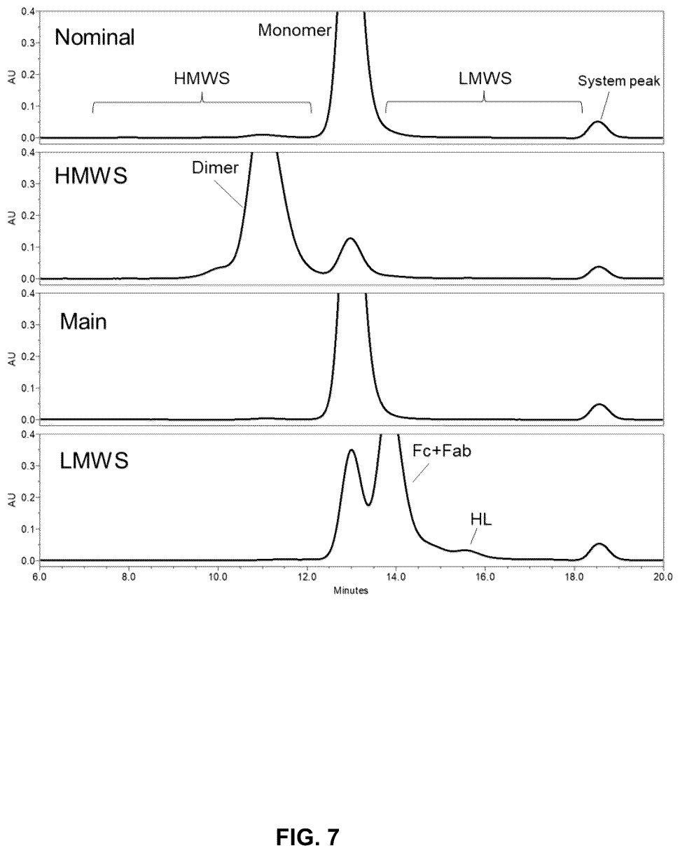

SE-HPLC chromatograms of nominal epcoritamab and the purified size fractions, analyzed by the SE-HPLC release method are shown in zoomed view in FIG. 7 and the relative purity of each fraction is shown below in Table 13.

TABLE 13
SE-HPLC purity of fractionated HMWS, monomer,
and LMWS epcoritamab size fractions

| | % Purity | | |
|---|---|---|---|
| Fraction | HMWS | Main | LMWS |
| Nominal | 1.6 | 98.4 | Not detected |
| HMWS | 85.0 | 14.5 | 0.5 |
| Monomer | 0.5 | 99.4 | 0.1 |
| LMWS | 0.4 | 32.4 | 67.2 |

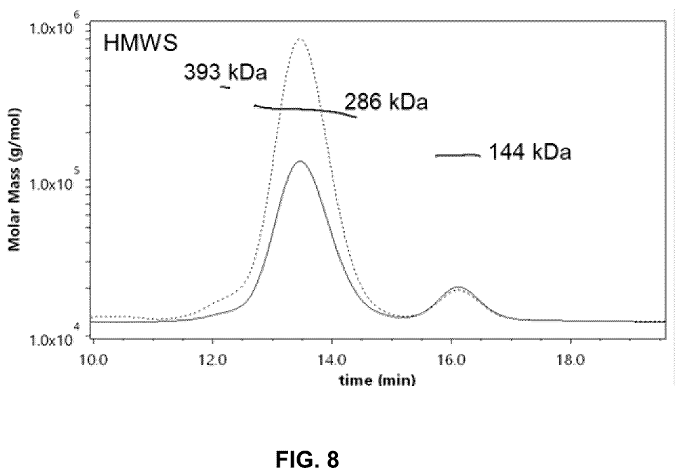

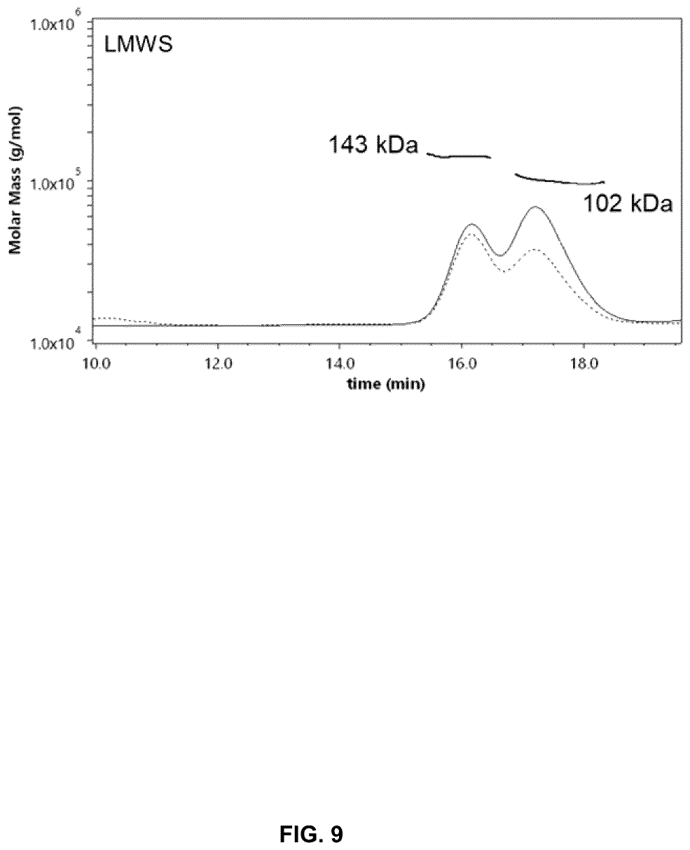

Peak identities for the purified fractions were established by multiple analyses. SEC-MALS data confirmed the predominant HMWS had a mass consistent with dimer and the predominant LMWS had a mass consistent with Fc+Fab fragment. These results are shown in FIG. 8 and FIG. 9, respectively.

The relationship between charge variants and size variants was characterized by icIEF and CIEX analysis of the size variants collected by semi-preparative SE-HPLC chromatography. HMWS and LMWS fractions primarily migrated as acidic variants using the icIEF method. The HMWS fraction separated both as acidic and basic forms using the CIEX method whereas LMWS mainly separated as acidic forms compared to the neutral peak but with one large form separating as a basic form.

These results are consistent with the non-reduced CE-SDS results of the CIEX fractions, in addition to SEC analysis of the CIEX fractions which showed enrichment of dimer and Fc+Fab fragment in the acidic fractions.

Thus, overall, epcoritamab was observed primarily as a single main peak corresponding to the monomeric antibody, whereas the HMWS were composed primarily of dimeric antibody with trace levels of earlier eluting oligomers. The LMWS consisted of Fc+Fab fragment species that were not resolved in the SE-HPLC method. SEC-MALS was used to confirm the peak identity and molecular weight of the monomer peak of nominal epcoritamab, as well as HMWS, under native conditions.

### Size Heterogeneity by Analytical Ultracentrifugation (SV-AUC)

The SV-AUC (sedimentation velocity analytical ultracentrifugation) method was used as an orthogonal method to SE-HPLC/SEC-MALS to assess the protein size and conformation directly from nominal epcoritamab in solution, with the primary goal of evaluating the abundance of HMWS and LMWS. The % peak area and sedimentation coefficients (s-value) of monomer, HMWS and LMWS are shown below in Table 14.

TABLE 14
SV-AUC Results

| Attribute | | Sediment Coefficient (s-value) | % Peak Area |
|---|---|---|---|
| LMWS | | 2.2 | 0.9 |
| Monomer | | 5.6 | 95.8 |
| HMWS | Dimer | 8.1 | 2.4 |
| | Oligomer | 10.0 | 0.8 |
| | | 15.0 | 0.2 |

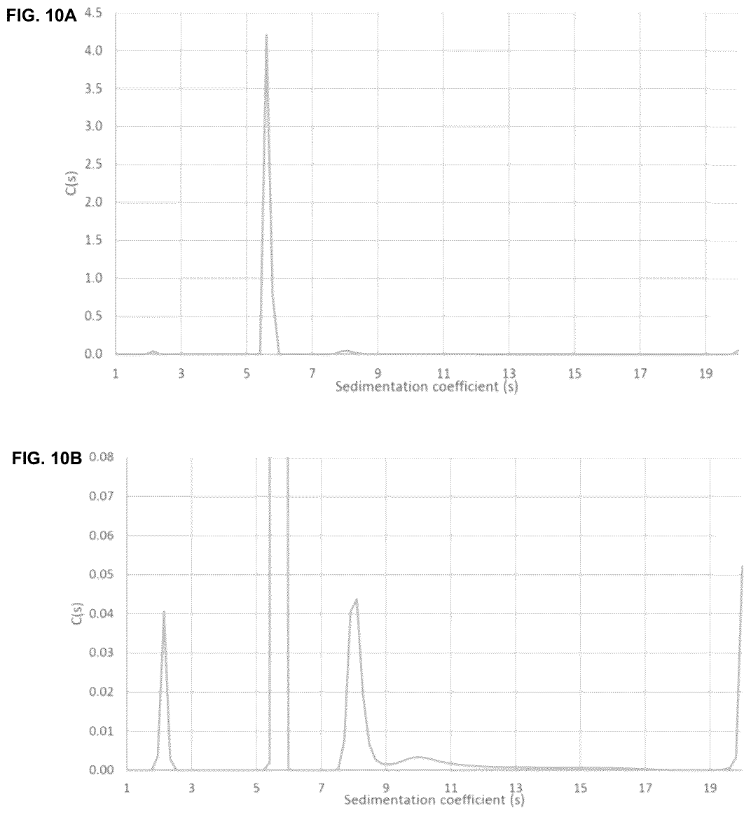

Distribution of species is shown in FIG. 10A-10B. Both the SE-HPLC and the SV-AUC method detected epcoritamab monomer as the major component. In general, the SV-AUC and the SE-HPLC assay detected a similar distribution of species, with SV-AUC detecting slightly higher levels of aggregates and fragments.

### Mass Heterogeneity by Non-Reduced and Reduced CE-SDS

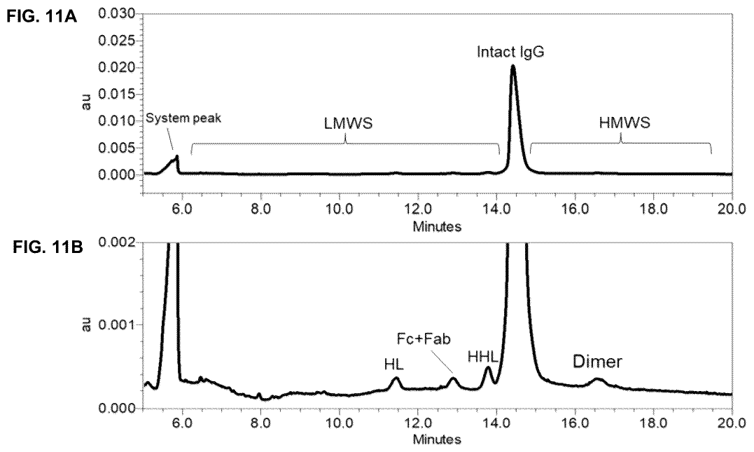

Mass heterogeneity was also assessed by non-reduced and reduced CE-SDS methods. The CE-SDS electropherogram of intact epcoritamab under non-reducing conditions is shown in FIGS. 11A-11B and the relative peak areas of individual species and calculated percent purities are shown below in Table 15.

TABLE 15
Non-reduced CE-SDS results

| Species | % Time Corrected Peak Area |
|---|---|
| IgG | 95.6 |
| HMWS | 0.9 |
| LMWS | 3.5 |

The most abundant species in the electropherogram is covalently associated monomer (IgG), which comprises 95.6% of the integrated peak area. Low levels of LMWS were observed and comprised HL, HHL as well as an Fc+Fab fragment (wherein H=heavy chain and L=light chain). Covalently associated dimer was observed in low abundance. Peak annotations were inferred from mass spectrometric analysis of the nominal material, the enriched size fractions as well as prior knowledge.

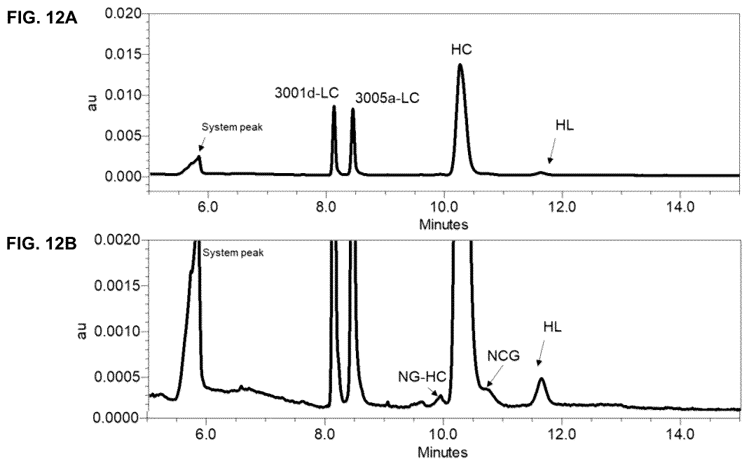

The CE-SDS electropherogram of reduced epcoritamab is shown in FIGS. 12A-12B and the calculated percent peak areas and purities are shown below in Table 16.

TABLE 16
Reduced CE-SDS results

| | % Time Corrected |
|---|---|
| Species | Peak Area |
| Purity (3005a HC + 3001d HC + 3001d LC + | 97.1 |
| 3005a LC) | |
| HC (3005a HC + 3001 HC) | 64.8 |
| NG-HC | 0.4 |
| NCG (3005a HC) | 1.0 |
| LC (3001d) | 15.7 |
| LC (3005a) | 16.6 |
| HMWS (including HL and NCG) | 2.1 |
| LMWS (including NG-HC) | 0.8 |

The CE-SDS electropherogram of reduced epcoritamab contained three major peaks: epcoritamab light chain (from 3001d), epcoritamab light chain (from 3005a) and epcoritamab heavy chains (combined peak from 3001d HC and 3005a HC). Non-glycosylated heavy chain (NG-HC) migrated as a minor peak immediately before the main heavy chain peak. The non-consensus glycosylation peak (NCG) was observed as a minor post-HC peak. The HL peak was identified by mass spectrometry to be a non-reducible thio-ether linkage-containing peak.

The identities of these peaks were established based on co-migration with samples of known composition.

### Acidic Species

Specific acidic variants (deamidated forms) of epcoritamab DS also have been assessed to be product-related impurities due to their impact on the biological activity. Certain variants, and effects on T cell activation ability of epcoritamab, are described in detail in Example 1, in the subsection entitled “Acidic Species and Effect on Biological Activity of Epcoritamab.” Additional variants, and effects on T cell activation ability of epcoritamab are described in detail below in the subsection entitled “Charge Variants.”

### High Molecular Weight Species

HMWS are multimers/protein aggregates typically formed by non-covalent and/or covalent association of monomeric proteins. Protein multimers/aggregates pose a potential patient risk in terms of immunogenicity. The predominant epcoritamab DS HMWS has been characterized as a dimer composed of associated monomeric antibodies.

Process mapping studies demonstrate that the cation exchange chromatography removes HMWS. The amount of HMWS was tested at release using a validated SE-HPLC method. The batch analysis results are shown below in Table 17.

TABLE 17
Batch analysis results for HMWS - SE-HPLC

| Epcoritamab batch | HMWS (%) |
|---|---|
| 1 | 1.6 |
| 2 | 1.5 |
| 3 | 1.7 |
| 4 | 1.9 |
| 5 | 1.8 |
| 6 | 1.9 |
| 7 | 1.9 |
| 8 | 2.0 |

As demonstrated by the results in Table 17, epcoritamab DS batch analysis showed that the level of HMWS is low (2.0% or less in all samples tested) and is consistent across the commercial process batches, including the PPQ batches, indicating that the process is well controlled and robust.

Semi-preparative purification of the epcoritamab HMWS resulted in an 85% pure sample with a retention time comparable to that of the unfractionated dimer peak in the nominal material. The mass of the purified HMWS was determined to be 286 kDa by SEC-MALS, which agreed with the mass of two antibodies containing the dominant glycoform (GIF/G2F). During the fractionation process it was observed that a small proportion of the dimer dissociated to monomer, suggesting that a fraction of the dimer is composed of non-covalently associated monomer. Care was taken to minimize dimer dissociation throughout the purification process and during sample analysis.

The purified HMWS was also analyzed by LC-MS using intact and partially reduced samples. Mass deconvolution of the intact dimer peak showed a dominant mass consistent with intact antibody, while mass deconvolution of the partially reduced dimer exhibited masses consistent with the two light chains and the two heavy chain. The predicted and observed masses for these species observed in the nominal and purified dimer are shown below in Table 18.

TABLE 18
LC-MS species observed for intact and
partially reduced HMWS/dimer fraction

| Species | Theoretical mass ¹ | Observed mass |
|---|---|---|
| (HMWS/dimer fraction) | (Da) | (Da) |
| Intact epcoritamab | 145,628 | 145,621 |
| Light Chain (3001d) | 23,438 | 23,437 |
| Light Chain (3005a) | 22,602 | 22,601 |
| Heavy Chain (3001d) | 49,584 | 49,583 |
| Heavy Chain (3005a) | 50,014 | 50,012 |

¹ Deglycosylated

Peptide mapping of the HMWS fraction furthermore showed enrichment of epcoritamab containing the LCTGSEA (SEQ ID NO: 9) (3005a HC) signal peptide in the HMWS fraction suggesting that the cysteine in the LCTGSEA (SEQ ID NO: 9) sequence might be involved in the disulfide-bonds leading to dimer formation. Therefore, the LCTGSEA residual signal peptide (SEQ ID NO: 9) is assessed to be an epcoritamab critical quality attribute (CQA).

CE-SDS under reducing conditions was used to quantify the amount of non-reducible species present. The HMWS fraction was found with elevated levels of non-reducible species compared to the nominal antibody. CE-SDS under non-reducing conditions was used to determine the size distribution of covalently associated species of the dimer. Covalent dimer migrates after the Intact IgG peak. The observed monomer peak in the HMWS fraction is due to dissociation of non-covalent dimer under the denaturing method conditions as well as a potential small amount of monomer present in the purified dimer fraction. Without accounting for the purity of the HMWS fraction, the covalent dimer comprising the LCTGSEA signal sequence (SEQ ID NO: 9) was calculated by CE-SDS to be approximately 57% of the HMWS fraction.

The biological activity of the HMWS fraction was measured using the T-cell activation assay (as described in Example 1) and found to be severely impacted (31% as compared to epcoritamab activity). The HMWS fraction was furthermore spiked into the nominal material at target levels ranging from 1% to 10% and submitted for biological activity analysis. The data are shown below in Table 19.

TABLE 19
Biological activity results for HMWS fraction

| | Actual % HMWS | % Relative |
|---|---|---|
| Sample | (SE-HPLC) | Biological Activity |
| Nominal | 1.6% | 94 |
| 2% HMWS | 2.4% | 107 |
| 4% HMWS | 3.4% | 107 |
| 6% HMWS | 4.8% | 98 |
| 8% HMWS | 6.0% | 100 |
| 10% HMWS | 7.4% | 96 |

The data presented in Table 19 showed that spike levels up to 7.4% did not impact the biological activity compared to the nominal epcoritamab material. However, due to its potential immunogenic impact, HMWS is assessed to be a CQA.

### Low Molecular Weight Species

LMWS are low molecular weight species typically derived from chemical or enzymatic cleavage at the hinge region as well as other sites in a monoclonal antibody and/or incomplete reoxidation of disulfide bonds in a bispecific antibody. The predominant LMWS present in epcoritamab DS has been characterized as the hinge-cleavage fragment containing Fc+Fab (antibody lacking a single Fab arm).

The amount of LMWS was tested at release using a non-reduced CE-SDS method. The batch analysis results are shown below in Table 20.

TABLE 20
Batch analysis results for LMWS - non-reduced CE-SDS

| Epcoritamab batch | LMWS (%) |
|---|---|
| 1 | 3.4 |
| 2 | 3.0 |
| 3 | 3.1 |
| 4 | 3.0 |
| 5 | 3.4 |
| 6 | 3.2 |
| 7 | 3.0 |
| 8 | 3.1 |

As demonstrated by the results in Table 20, epcoritamab DS batch analysis showed that the level of LMWS is low (3.4% or less in all samples tested) and is consistent across the commercial process batches, including the PPQ batches, indicating that the process is well controlled and robust.

Levels of LMWS were also analyzed using the reduced CD-SDS method. As shown in FIG. 12B and Table 16, levels of LMWS (including NG-HC) also were found to be low by this method.

The most abundant LMWS species was isolated in high purity (60.7% by SE-HPLC) and its retention time was confirmed against the nominal material. The mass of the purified LMWS variant was calculated to be 102 kDa by SEC-MALS, which is consistent with a single-arm antibody (Fc+Fab) resulting from the loss of a single Fab following heavy chain cleavage.

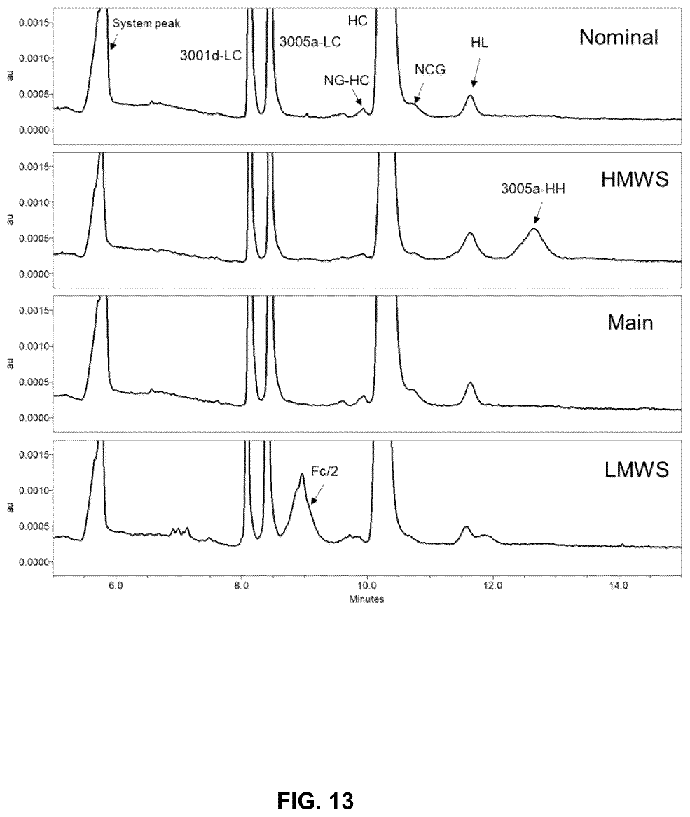

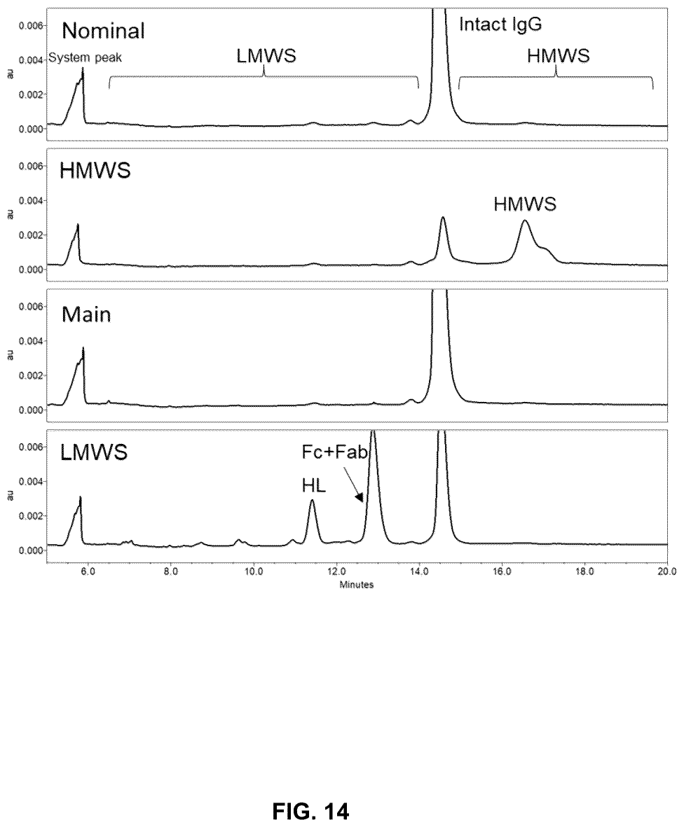

The isolated size variants were further characterized by reduced and non-reduced CE-SDS, the results of which are shown in FIG. 13 and FIG. 14, respectively. The structure of the 102 kDa LMWS Fc+Fab variant was confirmed by non-reduced CE-SDS and reversed phase LC-MS (intact and reduced) of the fraction to identify the points of cleavage. Intact mass analysis results confirmed the presence of Fc+Fab fragments, as well as a smaller amount of half-antibody (HL) confirming the results of the non-reduced CE-SDS result. The primary cleavages occurred at two different sites on both two heavy chains with lower-level cleavages also observed indicating that the Fc+Fab species are heterogeneous. These modifications are consistent with known IgG1 degradation pathways that lead to the creation of Fc+Fab and have been well documented in the literature. Characterization analysis of the corresponding Fab fragment could not be performed due to low abundance in the nominal material, and/or insufficient recovery during fractionation.

The biological activity of the LMWS fraction was measured using the T-cell activation assay (described in Example 1) and found to be severely impacted (40%) explained by the loss of one of antigen binding arms in the Fc+Fab species. Thus, LMWS as a group covering both disulfide-bond variants (such as HL and HHL) and backbone fragmentation (such as hinge fragmentation) is a critical quality attribute (CQA).

### Charge Variants

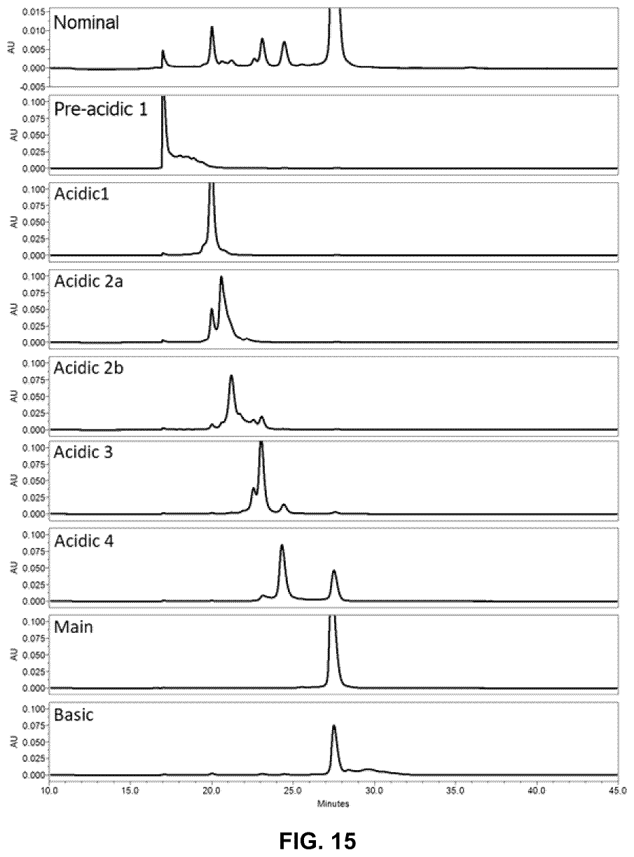

CIEX-HPLC as a semi-preparative technique was used to isolate epcoritamab charge variants utilizing the principals of cation exchange separation. Charge-based fractionation by CIEX-HPLC was performed with the CIEX-HPLC charge profile consisting of a main peak, a minor basic peak, and an acidic region consisting of several peaks of different abundance. The fractionation and collection of the peaks was performed with multiple injections and automatic fractionation collection. The basic fraction was only partly purified due to the low abundance of the species in the nominal material. FIG. 15 shows chromatograms from the CIEX-HPLC analysis of the charge fractions obtained by the semi-preparative CIEX-HPLC. LC-MS based peptide mapping was utilized to identify the post-translational modifications (PTMs) in the individual peaks/fractions and the most abundant species per fraction is shown below in Table 21.

TABLE 21
Relative, estimated abundance of major PTMs in charge fractions

| | | Pre- | Acidic | Acidic | Acidic | Acidic | Acidic | | |
|---|---|---|---|---|---|---|---|---|---|
| PTMs | Nominal | acidic 1 | 1 | 2a | 2b | 3 | 4 | Main | Basic |
| N-term E1-SEA | 17.7 | 42.9 | 13.0 | 26.9 | 12.1 | 24.7 | 74.5 | 0.7 | 9.8 |
| (3005a HC) | | | | | | | | | |
| N-term E1-LCTGSEA | 1.2 | 1.6 | 0.4 | 0.9 | 1.7 | 1.2 | 0.7 | 0.5 | 17.8 |
| (3005a HC) | | | | | | | | | |
| N103-Deamidated | 6.9 | 49.9 | 85.3 | 29.6 | 5.8 | 1.1 | 0.7 | 0.2 | 3.4 |
| (3005a HC) | | | | | | | | | |
| N106-Deamidated | 6.4 | 15.1 | 5.6 | 22.6 | 61.4 | 76 | 2.7 | 0.5 | 3.4 |
| (3005a HC) | | | | | | | | | |
| N103/N106-G2F | 0.1 | 0.1 | 0 | 1.3 | 3.4 | 0.1 | 0 | 0 | 0 |
| (3005a HC) | | | | | | | | | |
| N103/N106-A1F | 0.2 | 0.2 | 0.3 | 15.0 | 1.3 | 0 | 0 | 0 | 0.1 |
| (3005a HC) | | | | | | | | | |

### Pre-Acidic 1 Fraction—Characteristics and Biological Activity

Peptide mapping identified the main modification to be the N103 deamidated species (3005a HC) in combination with the residual E1 Ser-Glu-Ala (SEA) (3005a HC) signaling peptide. Intact mass analysis furthermore identified trace levels of a 3005a HC fragment matching fragmentation around the N103/N106 sites. The fragment was observed as a pre-HC LMW peak in reduced CE-SDS analysis. The fragment was observed to increase under heat.

As shown below in Table 22, the biological activity of the pre-acidic 1 fraction was measured using the T-cell activation assay and found to be severely impacted (<10%), likely due to the presence of significant levels of the N103 deamidation (3005a HC) and fragmentation of the 3005a arm that is directly involved in CD3 antigen binding. This observation was further supported by the crystal structure of the 3005a arm showing the proximity of the N103 deamidation site to the antigen binding site. The deamidation of N103 was therefore assessed to be a CQA of epcoritamab due to the detrimental impact on the biological activity of epcoritamab.

### Acidic 1 Fraction—Characteristics and Biological Activity

Peptide mapping identified the main modification to be the N103 deamidated (3005a) species and it furthermore showed presence of the E1 HC (3005a) SEA signal peptide.

As shown below in Table 22, similar to the pre-acidic 1 fraction, the biological activity of the acidic 1 fraction was measured and found to be severely impacted (<10%) likely due to the presence of significant levels of N103 deamidation in the 3005a HC CDR-region.

### Acidic 2a and 2b Fractions—Characteristics and Biological Activity

Peptide mapping of fraction acidic 2a identified the N103 HC (3005a) deamidation as the main component in combination with other modifications including N106 HC (3005a) deamidation, N106 HC (3005a) NCG (A1F) and E1 HC (3005a) residual SEA signal peptide. This was supported with intact mass data that confirmed the presence of the NCG (A1F) and the residual SEA signal peptide.

Peptide mapping of the acidic 2b fraction identified the N106 HC (3005a) deamidation as the main component in combination with other modifications including N103 HC (3005a) deamidation, N106 HC (3005a) NCG (A1F/G2F) and E1 HC (3005a) residual SEA signal peptide. This was supported with intact mass data that confirmed the presence of the NCG (A1F/G2F) and the residual SEA signal peptide.

The peptide mapping data of the 2a and 2b fractions support the observation of heterogenous species identified in the CIEX-HPLC and icIEF analysis of the fractions.

As shown below in Table 22, the biological activity of the acidic 2a and 2b fractions was measured using the T-cell activation assay and it was severely impacted (<30%) likely due to the combination of modifications of N103 deamidation and N106 deamidation in the 3005a HC CDR-region together with the N106 (3005a) NCG.

Due to the impact of the NCG on biological activity, it is assessed to be a CQA that is monitored by reduced CE-SDS as it elutes as a post-HC peak in the reduced CE-SDS method.

### Acidic 3 Fraction Characteristics and Biological Activity

Peptide mapping of the acidic 3 fraction identified the N106 HC (3005a) deamidation as the main component with trace levels of the N103 HC (3005a) deamidation.

As shown below in Table 22, the biological activity of the acidic 3 fraction was measured and was found not to be impacted (76%). This difference in biological activity, compared to the previous acidic fractions, is likely due to the spatial position of the N106 deamidation not directly involved in the epitope interaction, as observed in the crystal structure of the 3005a and CD3 binding interface. Though the isolated N106 deamidation event seemingly does not impact the biological activity it is still, however, assessed to be a CQA as the combination of the N106 deamidation with other modifications (especially the N103 deamidation as seen above) severely impacts the biological activity.

### Acidic 4 Fraction—Characteristics and Biological Activity

Peptide mapping of fraction 4 identified the E1 SEA (3005a HC) signal peptide as the main component with trace levels of both the N103 and N106 deamidations (3005a HC).

As shown below in Table 22, the biological activity of the acidic 4 fraction was measured using the T-cell activation assay and was found not to be impacted (95%). This is due to the SEA signal peptide not being part of the CDR and thus, the SEA residual signal peptide is a non-CQA.

### Basic Fraction—Characteristics and Biological Activity

The basic fraction was analyzed by SE-HPLC and showed an enrichment of multimer species consisting of IgG dimer (277 kDa) as determined by SEC-MALS. The presence of a covalent dimer in the basic fraction was furthermore observed in the non-reduced CE-SDS. Peptide mapping of the basic fraction confirmed the enrichment of epcoritamab containing the LCTGSEA (3005a HC) signal peptide (SEQ ID NO: 9) in the basic fraction. Reduced, intact mass analysis confirmed the presence of HH and HH with 2× the LCTGSEA signal peptide (SEQ ID NO: 9) suggesting that the cysteine in the LCTGSEA sequence (SEQ ID NO: 9) might be involved in the disulfide-bonds associated with the dimer formation. Therefore, the LCTGSEA residual signal peptide (SEQ ID NO: 9) is assessed to be a CQA.

As shown below in Table 22, the biological activity of the basic fraction was measured using the T-cell activation assay and the biological activity was only slightly impacted (71%). However, the significant levels of the main peak presence in the fraction might hinder a correct assessment of biological activity of the basic fraction.

### Main Peak Characteristics and Biological Activity

The analysis of the main peak by LC-MS peptide mapping shows no enrichment of N103/N106 deamidations, NCG, residual signaling peptides (SEA & LCTGSEA; SEQ ID NO: 9). The acidic peaks observed in icIEF for the main peak fraction are primarily due to the presence of sialylated N-glycosylated and glycated epcoritamab species.

As shown below in Table 22, the biological activity of the main peak fraction was measured using the T-cell activation assay with a trend showing increased activity (113%) relative to the nominal material. The increase in biological activity is likely due to the removal of modifications listed above that impact activity.

### Summary of Structure-Function Relationships of Epcoritamab Charge Variants

Reduced tryptic peptide mapping with mass spectrometry detection was performed to evaluate post-translational modifications present in the CIEX fractions of epcoritamab. Post-translational modifications, observed in the side-by-side analysis of the CIEX fractions are summarized below in Table 22. The biological activity, as measured in the T cell activation assay (see Example 1) was mainly impacted by the pre-acidic 1, acidic 1 and acidic 2a/2b fractions due to the presence of the 3005a HC N103/N106 deamidations, 3005a HC N-terminal fragments, and 3005a HC non-consensus glycosylations.

TABLE 22
Major identified charge variants of epcoritamab by CIEX

| | | Estimated PTM levels | Biological Activity^{a} |
|---|---|---|---|
| Fractions | Major identified components | (rel %) | (rel %) |
| Nominal | Deamidated N103 | See Error! Reference | 92 |
| | Deamidated N106 | source not found. | |
| | SEA residual signal peptide | | |
| Pre-acidic 1 | Deamidated N103 | 49.9 | 6 |
| | Deamidated N106 | 15.1 | |
| | SEA residual signal peptide | 42.9 | |
| Acidic 1 | Deamidated N103 | 85.3 | 9 |
| | SEA residual signal peptide | 13.0 | |
| Acidic 2a | Deamidation N103 | 29.6 | 25 |
| | Deamidation N106 | 22.6 | |
| | Non-consensus glycosylation (A1F) | 15.0 | |
| | SEA residual signal peptide | 26.9 | |
| Acidic 2b | Deamidation N106 | 61.4 | 28 |
| | Non-consensus glycosylation (G2F) | 3.4 | |
| | SEA residual signal peptide | 12.1 | |
| Acidic 3 | Deamidation N106 | 76.0 | 90 |
| Acidic 4 | SEA residual signal peptide | 74.5 | 95 |
| Main peak | Epcoritamab | n.d. | 113 |
| Basic | LCTGSEA residual signal peptide | 17.8 | 71 |
| | SEA residual signal peptide | 9.8 | |

^{a}Values <40% for information only as values are outside the qualified range of the assay

TABLE 23
SEQUENCE LISTING SUMMARY

| SEQ | | |
|---|---|---|
| ID | Description | Sequence |
| 1 | CD3 Binding Arm | EVKLVESGGGLVQPGGSLRLSCAASGFTFNTYAMNWVRQAPGKGLE |
| | 3005a Heavy Chain | WVARIRSKYNNYATYYADSVKDRFTISRDDSKSSLYLQMNNLKTEDTA |
| | | MYYCVRHGNFGNSYVSWFAYWGQGTLVTVSSASTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTCPPC |
| | | PAPEFEGGPSVFLFPPKPKDTLMISRTPEVTCVVVAVSHEDPEVKFNW |
| | | YVDGVEVHNAKTKPREEQYNSTYRVVSVLTVLHQDWLNGKEYKCKV |
| | | SNKALPAPIEKTISKAKGQPREPQVYTLPPSREEMTKNQVSLTCLVKGF |
| | | YPSDIAVEWESNGQPENNYKTTPPVLDSDGSFLLYSKLTVDKSRWQQ |
| | | GNVFSCSVMHEALHNHYTQKSLSLSPG |
| 2 | CD3 Binding Arm | QAVVTQEPSFSVSPGGTVTLTCRSSTGAVTTSNYANWVQQTPGQAF |
| | 3005a Light Chain | RGLIGGTNKRAPGVPARFSGSLIGDKAALTITGAQADDESIYFCALWYS |
| | | NLWVFGGGTKLTVLGQPKAAPSVTLFPPSSEELQANKATLVCLISDFY |
| | | PGAVTVAWKADSSPVKAGVETTTPSKQSNNKYAASSYLSLTPEQWKS |
| | | HRSYSCQVTHEGSTVEKTVAPTECS |
| 3 | CD20 Binding Arm | EVOLVESGGGLVQPDRSLRLSCAASGFTFHDYAMHWVRQAPGKGLE |
| | 3001d Heavy Chain | WVSTISWNSGTIGYADSVKGRFTISRDNAKNSLYLQMNSLRAEDTAL |
| | | YYCAKDIQYGNYYYGMDVWGQGTTVTVSSASTKGPSVFPLAPSSKST |
| | | SGGTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPCPA |
| | | PEFEGGPSVFLFPPKPKDTLMISRTPEVTCVVVAVSHEDPEVKFNWYV |
| | | DGVEVHNAKTKPREEQYNSTYRVVSVLTVLHQDWLNGKEYKCKVSN |
| | | KALPAPIEKTISKAKGQPREPQVYTLPPSREEMTKNQVSLTCLVKGFYP |
| | | SDIAVEWESNGQPENNYKTTPPVLDSDGSFFLYSRLTVDKSRWQQG |
| | | NVFSCSVMHEALHNHYTQKSLSLSPG |
| 4 | CD20 Binding Arm | EIVLTQSPATLSLSPGERATLSCRASQSVSSYLAWYQQKPGQAPRLLIY |
| | 3001d Light Chain | DASNRATGIPARFSGSGSGTDFTLTISSLEPEDFAVYYCQQRSNWPITF |
| | | GQGTRLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACE |
| | | VTHQGLSSPVTKSFNRGEC |
| 5 | CD3 Binding Arm | EVKLVESGGGLVQPGGSLRLSCAASGFTFNTYAMNWVRQAPGKGLE |
| | VH Region | WVARIRSKYNNYATYYADSVKDRFTISRDDSKSSLYLQMNNLKTEDTA |
| | | MYYCVRHGNFGNSYVSWFAYWGQGTLVTVSS |
| 6 | CD3 Binding Arm | QAVVTQEPSFSVSPGGTVTLTCRSSTGAVTTSNYANWVQQTPGQAF |
| | VL Region | RGLIGGTNKRAPGVPARFSGSLIGDKAALTITGAQADDESIYFCALWYS |
| | | NLWVFGGGTKLTVL |
| 7 | CD20 Binding Arm | EVOLVESGGGLVQPDRSLRLSCAASGFTFHDYAMHWVRQAPGKGLE |
| | VH Region | WVSTISWNSGTIGYADSVKGRFTISRDNAKNSLYLQMNSLRAEDTAL |
| | | YYCAKDIQYGNYYYGMDVWGQGTTVTVSS |
| 8 | CD20 Binding Arm | EIVLTQSPATLSLSPGERATLSCRASQSVSSYLAWYQQKPGQAPRLLIY |
| | VL Region | DASNRATGIPARFSGSGSGTDFTLTISSLEPEDFAVYYCQQRSNWPITF |
| | | GQGTRLEIK |
| 9 | Signal Peptide | LCTGSEA |

1. A composition comprising at least 95% epcoritamab and less than 5% of an impurity, wherein epcoritamab comprises: (i) a CD3 binding arm comprising a heavy chain sequence as shown in SEQ ID NO: 1 and a light chain sequence as shown in SEQ ID NO: 2; and (ii) a CD20 binding arm comprising a heavy chain sequence as shown in SEQ ID NO: 3 and a light chain sequence as shown in SEQ ID NO: 4.

2. (canceled)

3. The composition of claim 1, wherein the composition comprises less than 3% of the impurity.

4. The composition of claim 1, wherein the impurity is a product-related impurity.

5. The composition of claim 4, wherein the product-related impurity is selected from the group consisting of a high molecular weight species (HMWS) of epcoritamab, a low molecular weight species (LMWS) of epcoritamab, a charge variant of epcoritamab, an acidic species of epcoritamab, a basic species of epcoritamab, a deamidated species of epcoritamab, a glycosylated species of epcoritamab, a glycated species of epcoritamab, an oxidized species of epcoritamab, an N-terminal modification of epcoritamab, an aggregate of epcoritamab, a fragment of epcoritamab, a residual signal peptide modification of epcoritamab, an Fc fragment of epcoritamab and a Fab fragment of epcoritamab.

6. (canceled)

7. The composition of claim 5, wherein the HMWS is an epcoritamab dimer comprising a signal sequence as shown in SEQ ID NO: 9.

8. (canceled)

9. The composition of claim 5, wherein the LWMS is an Fc+Fab fragment of epcoritamab.

10. (canceled)

11. The composition of claim 5, wherein the deamidated acidic species of epcoritamab comprises deamidation of one or more residues selected from the group consisting of a N103 residue in the heavy chain of the CD3 binding arm of epcoritamab, a N106 residue in the heavy chain of the CD3 binding arm of epcoritamab, a N103 and a N106 residue in the heavy chain of the CD3 binding arm of epcoritamab, a N333 residue in the heavy chain of the CD3 binding arm of epcoritamab, a N330 residue in the heavy chain of the CD20 binding arm chain of epcoritamab, and combinations thereof.

12. The composition of claim 1, wherein the impurity is a process-related impurity.

13. The composition of claim 12, wherein the process-related impurity is selected from the group consisting of a reducing agent, an oxidizing agent, a manufacturing intermediate, a residual IgG1 homodimer intermediate, a residual host cell protein, a host cell nucleic acid, a media component and a chromatographic material.

14. (canceled)

15. The composition of claim 13, wherein the reducing agent is 2-mercaptoethylamine (2-MEA).

16. The composition of claim 15, wherein 2-MEA is present in the composition at a level at least 10-fold lower than the Permitted Daily Exposure (PDE) for 2-MEA.

17. (canceled)

18. The composition of claim 13, wherein the oxidizing agent is dehydro-L-Ascorbic Acid (dhAA).

19. The composition of claim 13, wherein dhAA is present in the composition at a level at least 100-fold lower than the Permitted Daily Exposure (PDE) for dhAA.

20. (canceled)

21. The composition of claim 13, wherein the manufacturing intermediate is the 3005a antibody or a portion thereof.

22. The composition of claim 13, wherein the manufacturing intermediate is the 3001d antibody or a portion thereof.

23. (canceled)

24. The composition of claim 5, wherein the glycosylated species comprises glycosylation of one or more residues selected from the group consisting of a N106 residue in the heavy chain of the CD3 binding arm of epcoritamab, a N305 residue in the heavy chain of the CD3 binding arm of epcoritamab, a N302 residue in the heavy chain of the CD20 binding arm of epcoritamab, and combinations thereof.

25. (canceled)

26. The composition of claim 5, wherein the glycated species comprises glycation of one or more residues selected from the group consisting of a K331 residue in the heavy chain of the CD20 binding arm of epcoritamab, a K334 residue in the heavy chain of the CD3 binding arm chain of epcoritamab, and combinations thereof.

27. (canceled)

28. The composition of claim 5, wherein the oxidized species comprises oxidation of one or more residues selected from the group consisting of a M363 residue in the heavy chain of the CD20 binding arm of epcoritamab, a M366 residue in the heavy chain of the CD3 binding arm of epcoritamab, and combinations thereof.

29. (canceled)

30. The composition of claim 13, wherein the residual IgG1 homodimer intermediate is a residual 3005a species.

31. The composition of claim 13, wherein the residual IgG1 homodimer intermediate is a residual 3001d species.

32. The composition of claim 13, wherein the residual IgG1 homodimer intermediate is a mixture of a residual 3005a species and a residual 3001d species.

33. The composition of claim 13, wherein the composition comprises at least 95% epcoritamab and between 0%-2% of the residual IgG1 homodimer intermediate.

34. The composition of claim 13, wherein the process-related impurity is a residual HCP, and wherein the residual HCP is selected from the group consisting of a polyubiquitin, a ubiquitin, a IgG-binding protein A, a peroxiredoxin-1, a thioredoxin reductase 1 (cytoplasmic), a phospholipase B-like, and combinations thereof.

35. (canceled)

36. The composition of claim 34, wherein the composition comprises at least 95% epcoritamab and between 0-100 parts per million (ppm) of the residual HCP.

37. The composition of claim 34, wherein the residual HCP is between 0-50 ppm.

38. (canceled)

39. The composition of claim 5, wherein the N-terminal modification is selected from the group consisting of a glutamate to pyroglutamate conversion, a residual signal peptide attached to epcoritamab, and combinations thereof.

40. The composition of claim 39, wherein the glutamate to pyroglutamate conversion occurs in at least one of a heavy chain of the CD3 binding arm of epcoritamab, a heavy chain of the CD20 binding arm of epcoritamab and a light chain of the CD20 binding arm of epcoritamab.

41. The composition of claim 40, wherein the glutamine to pyroglutamate conversion occurs in the light chain of the CD3 binding arm of epcoritamab.

42. The composition of claim 39, wherein the residual signal peptide comprises an amino acid sequence selected from the group consisting of (a) Ser-Glu-Ala and (b) Leu-Cys-Thr-Gly-Ser-Glu-Ala (SEQ ID NO: 9).

43. A composition comprising at least 95% epcoritamab and less than 5% of an acidic epcoritamab variant that is deamidated at residues N103 and N106 of CD3 heavy chain of epcoritamab.

44. A composition comprising at least 95% epcoritamab and less than 5% of a high molecular weight species (HMWS) of epcoritamab, wherein the HMWS is an epcoritamab dimer comprising a signal sequence having the amino acid sequence shown in SEQ ID NO: 9.

45. A composition comprising at least 95% epcoritamab and less than 5% of a low molecular weight species (LMWS) of epcoritamab, wherein the LMWS is an Fc+Fab fragment of epcoritamab.

46. The composition of claim 1, wherein the composition is a pharmaceutical composition.

47. The composition of claim 46, wherein the composition is a stable liquid composition suitable for subcutaneous administration.

48. The composition of claim 47, wherein the composition is a stable liquid aqueous composition that has never been subjected to prior reconstitution.

49. The composition of claim 47, wherein the composition is a lyophilized composition which is subjected to reconstitution before subcutaneous administration

50. The composition of claim 48, wherein the composition comprises about 4 to 5 mg/mL of epcoritamab.

51. The composition of claim 48, wherein the composition comprises about 60 mg/mL of epcoritamab.

52. A method of preparing the composition of claim 1, the method comprising:
(a) combining a first binding arm of epcoritamab and a second binding arm of epcoritamab to form a combined sample;
(b) exposing the combined sample to reducing conditions to form a reduced combined sample;
(c) treating the reduced combined sample with an oxidizing agent to form an oxidized sample; and
(d) subjecting the oxidized sample to cation exchange chromatography such that the composition is prepared.

53. The method of claim 52, wherein the combined sample is pH-adjusted to pH 7.0-7.7 before exposure to the reducing conditions.

54. The method of claim 52, wherein ultrafiltration/diafiltration (UF/DF) is performed after each of steps (b), (c) and (d).


SEQUENCE LISTING (9 sequences)

SEQ ID NO: 1 - protein, 454 aa, synthetic construct
  REGION 1..454: note = Antibody heavy chain
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 2 - protein, 215 aa, synthetic construct
  REGION 1..215: note = Antibody light chain
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 451 aa, synthetic construct
  REGION 1..451: note = Antibody heavy chain
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 4 - protein, 214 aa, synthetic construct
  REGION 1..214: note = Antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 125 aa, synthetic construct
  REGION 1..125: note = Heavy chain variable region sequence l
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 6 - protein, 109 aa, synthetic construct
  REGION 1..109: note = Light chain variable region sequence
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 7 - protein, 122 aa, synthetic construct
  REGION 1..122: note = Heavy chain variable region sequence
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 8 - protein, 107 aa, synthetic construct
  REGION 1..107: note = Light chain variable region sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 9 - protein, 7 aa, synthetic construct
LCTGSEA